(19) United States
(12) Patent Application Publication
(10) Pub. No.: US 2022/0415478 A1
(43) Pub. Date: Dec. 29, 2022

(54) SYSTEMS AND METHODS FOR MENTAL EXERCISES AND IMPROVED COGNITION

(71) Applicant: Think Now, Inc., San Francisco, CA (US)

(72) Inventors: Gregory V. Simpson, San Anselmo, CA (US); Agatha Lenartowicz, Pasadena, CA (US); Brett G. DeSchepper, Albany, CA (US)

(21) Appl. No.: 17/842,656

(22) Filed: Jun. 16, 2022

Related U.S. Application Data
(60) Provisional application No. 63/216,448, filed on Jun. 29, 2021.

(51) Int. Cl. G16H 20/70; A61B 5/369; A61B 5/16

(52) U.S. Cl. CPC G16H 20/70; A61B 5/369; A61B 5/165

(57) ABSTRACT

Systems and methods for improved cognition via mental exercises are provided. In some embodiments, contextual information regarding the user and her environment are collected. Subsequently, the user is presented with a cognitive task, wherein the cognitive task includes a stimuli and the task specifies a desired response to the stimuli. Feedback is then collected from the user. Additionally the exercise results are assessed. The feedback, results and context information are then all aggregated in order to generate guidance for the user of the system. Guidance may be determined by a rule based system, via AI modeling, or by some combination of the two.

### CROSS REFERENCE TO RELATED APPLICATION

This non-provisional application claims the benefit of U.S. Provisional Application No. 63/216,448 filed on Jun. 29, 2021, of the same title, pending, which application is incorporated herein in its entirety by this reference.

### BACKGROUND

The present invention relates in general to the field of cognitive training, and more specifically to a computer program and systems for cognitive training techniques for improving cognition in a myriad of ways. Such systems and methods are useful to normal populations as well as individuals with a wide-range of cognitive disorders including but not limited to stress, anxiety, social anxiety disorder, depression, ADHD, Autism-Asberger's, Schizophrenia, TBI, PTSD and stroke.

Our everyday activities also drive cognitive changes. Those activities might be intentional, like learning how to play tennis or how to speak a new language, but also unintentional such as the formation of habits. It is the intention of our training to bring a specific set of skills related to attention and attentional control back to a normal range of intentional control with the goal to enhance health and performance.

Cognitive training exists in many different forms including simple practice and repetition of activity with intentional goals in a therapeutic or coaching setting to computer directed tasks that are highly controlled, systematic, and adaptive to the individual. Tasks are any set of stimuli and related cognitive activities that require cognitive or behavioral responses by an individual. With the advent of mobile computing systems, many software programs for cognitive training are now available to individuals as they progress through their day, also known as in situ availability.

Many cognitive training programs focus on specific recognition tasks or memory tasks. Focus is the quality of a person's attentional control to appropriately accomplish a task. It is the objective of this invention to train higher level of cognitive skills commonly referred to as executive function. Executive function knowledge and awareness are required for improving these processes through training.

Brain training methods use repetitive tasks to drive changes in neural responses, without explicit learning, that can impact future performance. Cognitive training, as opposed to brain training, relates to how learning through concepts and experiences cause change in the neurology and behavior of individuals. Neuroscience has shown that such guided learning activities can have significant impact on the human maladies and performance. That impact not only appears on pre- and post-tests administered to individuals to measure behavioral changes but also through modern imaging techniques such as fMRI and EEG.

Despite the significant advantages that cognitive training can have on an individual's life, it is generally still inaccessible to the population as a whole. Cognitive training has been relegated to the laboratory and clinical settings, which is generally costly or difficult to access.

It is therefore apparent that an urgent need exists for systems and methods for improved means of cognitive training that is more widespread and accessible by the general population. Such systems and methods are designed to provide cognitive training to alleviate disorders and improve functioning by helping users make guided enhancements of their cognitive abilities through increased awareness and consequent training of control based on that awareness. This invention is designed to both teach and train users how to increase self-awareness, and provide tools for the benefit of one's well-being.

### SUMMARY

The present systems and methods relate to improving cognitive functioning through guided training activities on a computerized device. Such systems and methods enable improvements in not just improved cognitive functioning when engaged in these cognitive exercises but transfer directly into real-life situations.

In some embodiments, contextual information regarding the user and her environment are collected. This context includes biometric and psychological data collected from the user, environmental conditions, date, and time. Subsequently, the user is presented with a cognitive task, wherein the cognitive task includes a stimuli and the task specifies a desired response to the stimuli. In some cases, the stimuli is presented as random inter-stimulus intervals (ISI), which are substantially between a reaction-reset interval and an attention-sustaining interval. The device used to present the cognitive task to the user may include any of a smartphone, a tablet, a personal computer, a VR headset, a smart TV and other home entertainment systems, an augmented reality headset and holographic projection systems. Additionally, the device could be integrated into physical exercise equipment and digital home entertainment and home control systems for lighting, heating and sleep systems. The device may also be used to present the cognitive task to multiple people at the same time and to incorporate both an individual user's performance and the performance of others in a group through group interfaces (e.g. digital theater), and may be implemented across multiple group interfaces around the world at the same time.

Feedback is then collected from the user. This feedback may include free-form text notes and other collected data (such as EEG data, facial recognition/emotion data, pupil dilation, heart rate and heart rate variability data, respiratory data, body temperature data, electrodermal response data, etc.). Additionally, the exercise results are assessed. The feedback, results and context information are then all aggregated in order to generate guidance for the user of the system. Guidance may be determined by a rule based system, via AI modeling (of both individual and group data), or by some combination of the two. The guidance includes resource documents and progression to an advanced cognitive task.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 is a block diagram of a desktop computer, tablet, Smartphone, and laptop connected through the internet to remote servers, in accordance with some embodiment;

FIG. 2 is a block diagram showing a system and method for training Meta-attention, in accordance with some embodiment;

FIG. 3 is a block diagram showing a system and method of non-compelling attention training, in accordance with some embodiment;

FIG. 4 is a block diagram showing a system and method for continuous performance during attention training, in accordance with some embodiment;

FIG. 5 is a block diagram showing a system and method for inhibition of response in a cognitive task, in accordance with some embodiment;

FIG. 6 is a block diagram showing a system and method of low variance of response time in a cognitive task, in accordance with some embodiment;

FIG. 7 is a block diagram showing a system and method of timing specificity in a cognitive task, in accordance with some embodiment;

FIG. 8 is a block diagram showing a system and method for determining the feedback for a cognitive task, in accordance with some embodiment;

FIG. 9 is a block diagram showing a system and method for evaluating the inhibition of response results from a cognitive task, in accordance with some embodiment;

FIG. 10 is a block diagram showing a system and method for evaluating the low variance of response results from a cognitive task, in accordance with some embodiment;

FIG. 11 is a block diagram showing a system and method for evaluating the timing specificity results from a cognitive task, in accordance with some embodiment;

FIG. 12 is simulated screen shots of the inhibition of response in a cognitive task, in accordance with some embodiments;

FIG. 13 is simulated screen shots of the low variance of response time in a cognitive task, in accordance with some embodiments;

FIG. 14 is simulated screenshots of the timing specificity in a cognitive task, in accordance with some embodiments;

FIG. 15 is a top view of a user's head with exemplary locations for measuring SSVEP and/or SSAEP, in accordance with some embodiments;

FIGS. 16A-16C and 17A-17C are screenshots illustrating exemplary protocols for the embodiment of the cognitive measuring system, in accordance with some embodiments;

FIGS. 18A-18G are example interfaces for the cognitive measuring system, in accordance with some embodiments;

FIGS. 19A and 19B are example use cases for the cognitive measuring system, in accordance with some embodiments;

FIG. 20 is an example of the cognitive measuring system, in accordance with some embodiments;

FIG. 21 is an example block diagram of the user devices employed in the cognitive measuring system, in accordance with some embodiments;

FIG. 22 is an example block diagram of the training application on the user device, in accordance with some embodiments;

FIG. 23 is an illustration of an example process for cognitive training, in accordance with some embodiments;

FIG. 24 is an illustration of an example process for exercise completion, in accordance with some embodiments;

FIG. 25 is an illustration of an example process of exercise selection, in accordance with some embodiments;

FIG. 26 is an illustration of an example process of the exercise, in accordance with some embodiments;

FIG. 27 is an illustration of an example process for running the cognitive exercise, in accordance with some embodiments;

FIGS. 28A and 28B are illustrations of example screenshots of the cognitive training, in accordance with some embodiments;

FIG. 29A is an illustration of an example for the framework for improving cognitive functioning, in accordance with some embodiments;

FIG. 29B is an illustration of an example for the loop involved with transferring of learnings into real life, in accordance with some embodiments; and

FIGS. 30A and 30B are illustrations of computer systems capable of implementing the cognitive training system, in accordance with some embodiments.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “always,” “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary. Conversely, terms such as “can” or “may” are used interchangeably and are intended to describe alternative and/or optional features, i.e., may not be necessary or preferred, for the disclosed embodiments.

The present invention relates to systems and methods for training an individual's cognitive abilities. In some embodiments, attention will focus on cognitive tasks that are designed to improve a person's Meta-attention and attentional control. In yet other embodiments, the guided training may be more widely applicable, focusing on a greater selection of cognitive functions. The training programs uses the techniques enumerated below to drive a user to experience all forms of attentional state change and control. Other tasks include tailored guidance and feedback to the user in order to maximize cognitive enhancements. The user's performance can be monitored through real-time and/or periodically and feedback can be provided relative to the user's history and/or compared to other users.

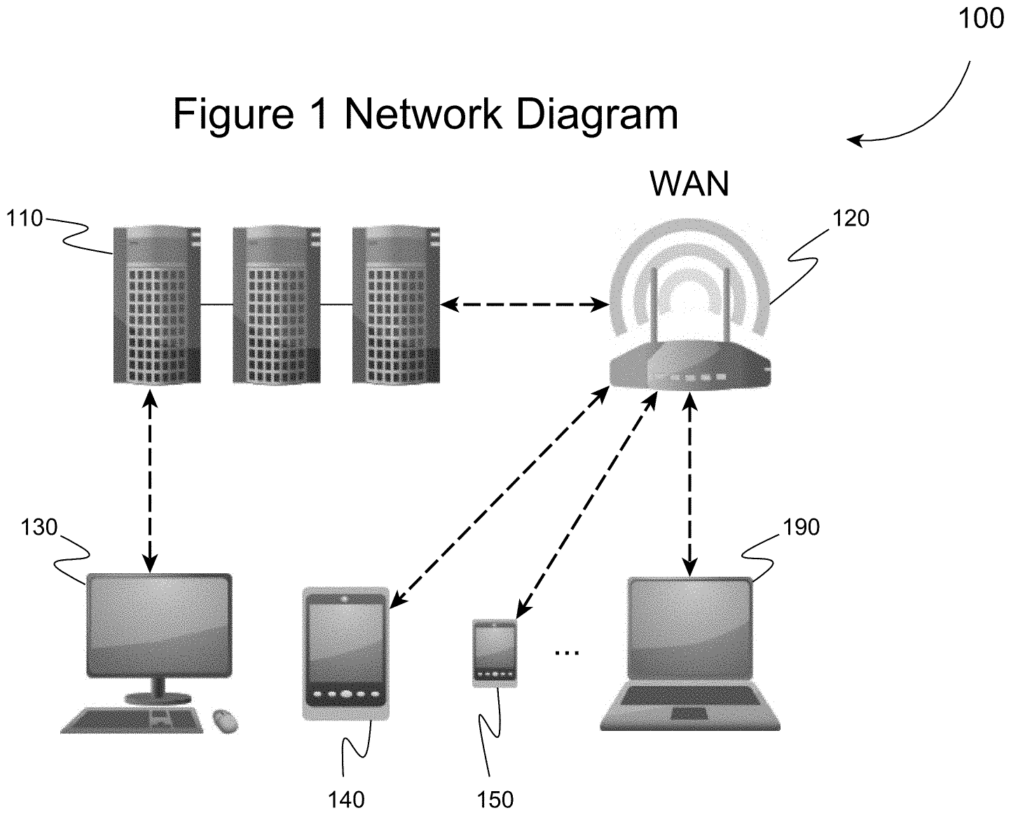

Referring to FIG. 1, a training system 100 is shown for executing a computer program to train, or retrain a cognitive training program according to the some embodiments. The training system 100 contains a server 110, which allows a user to remotely access the cognitive tasks and results over the cloud. The servers 110 maybe connected through local area network (LAN) (not shown), a wide area network (WAN) 120 or via modern connections, directly or through the internet; however, connection to the internet or a server is not required for the invention to be effective, in some embodiments. The user may use any of the following devices but training is not limited to the following: 1) a desktop computer system 130, 2) a tablet computer 140, 3) Smartphone or other small portable handheld device 150, and 4) laptop computer 160. The desktop computer 130 contains a mouse, keyboard, and monitor attached to a computer containing a computer processing unit (CPU), hard disk, and CD ROM (not shown). The laptop computer 160 contains a display screen, internal CPU, keyboard, and touchpad. The monitor in 130 and displays screens of 140, 150, and 160 allow the user to see the stimuli. The mouse in 130 and touchpad of 160 allow the user to select the stimuli according to the specific cognitive task. While the display screens of the tablet computer 140 and handheld device 150 allow the user to directly select the stimuli according to the specific cognitive task. The computer network 100 allows information such as the training, test scores, statistics, and other information to flow from any device 130, 140, 150, and 160 to a server 110. Although a number of different computer platforms are applicable to some embodiments, in some cases the systems execute on tablet computers or handheld devices running any operating system.

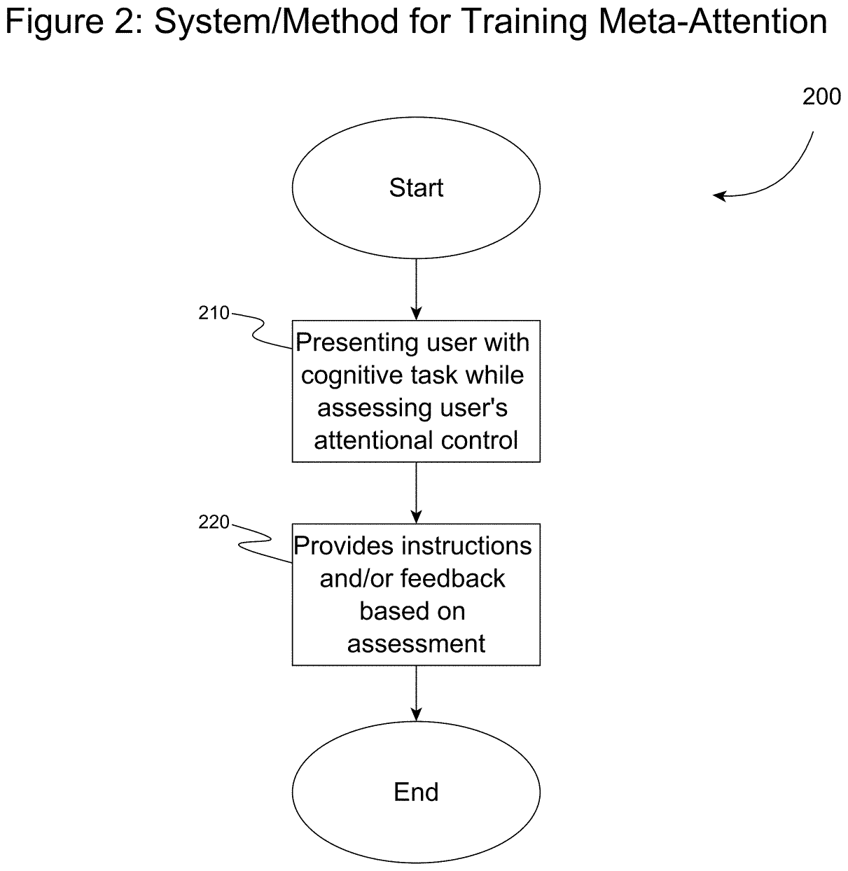

Now referring to FIG. 2, the block diagram shows a system and method for attaining Meta-attention 200. Meta-attention is defined as knowledge of the factors that influence one's attention and an awareness of one's attentional processes as they occur. Such knowledge and/or awareness of attention are required for improving these processes through training. Meta-attention is trained through being presented with a cognitive task while the training system assesses the user's attentional control 210 and receiving instructions and/or feedback 220. The assessment of the user's attentional control can occur continuously or periodically. Moreover, the non-compelling cognitive tasks 210 are any type of computer controlled and goal driven training tasks that require the user to focus on a cognitive task according to the prescribed rules. The instructions and/or feedback results 220 are the reactions and responses to a particular non-compelling cognitive task. Moreover, the instructions and/or feedback results 220 can include, but are not limited to real-time scoring, post-training play scoring, historical statistics, and guidance for reflecting on the meaning of the feedback with respect to meta-attention.

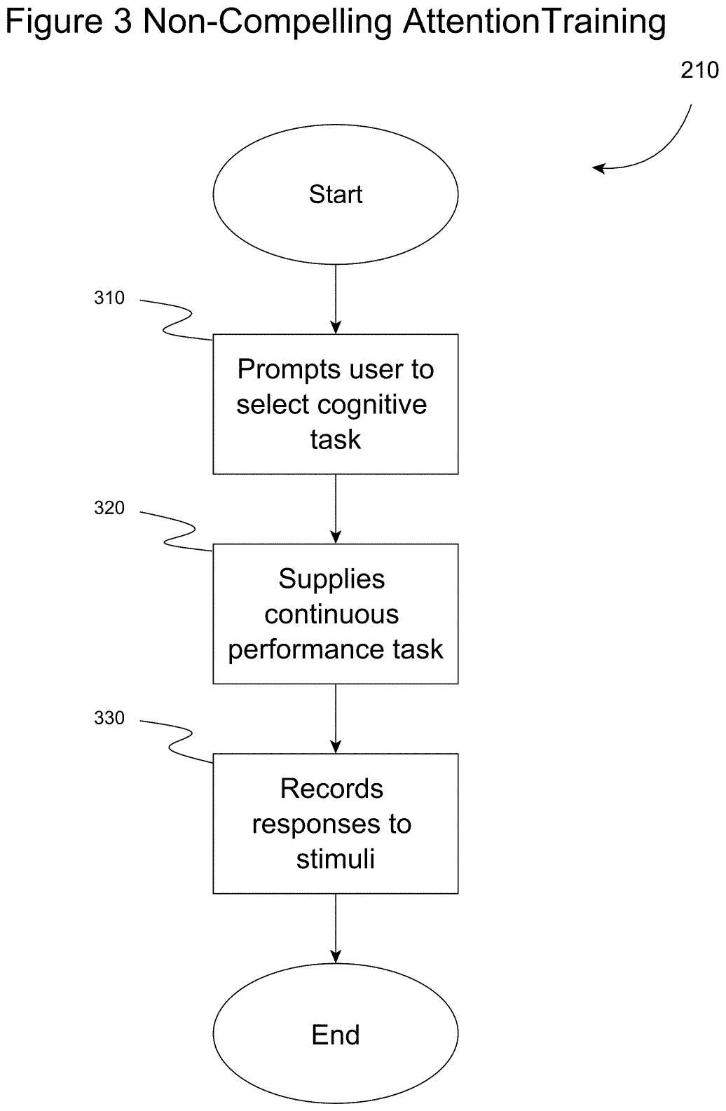

FIG. 3 is a block diagram detailing a non-compelling cognitive task 210. Each non-compelling cognitive task proceeds in the same fashion, 1) the user is prompted to select a cognitive task 310, 2) a continuous performance task is presented to the user 320, and 3) the responses to the stimuli are recorded 330. The cognitive tasks 310 are any computer controlled, goal driven training task. The cognitive tasks 310 are computer controlled meaning they run on any of the computer platforms described above in FIG. 1. The cognitive tasks 310 are also goal driven. The activity produces a score that represents to the user their skill at attentional control and then links that score to the user's desire to be in better control of their attention. The cognitive tasks 310 are best accomplished through continuous performance tasks 320. A continuous performance task 320 is one that does not allow the user to disengage from the cognitive task without incurring a performance/score decline. A continuous performance task 320 is important because it continuously engages the parts of the brain that are central to the Meta-attention training.

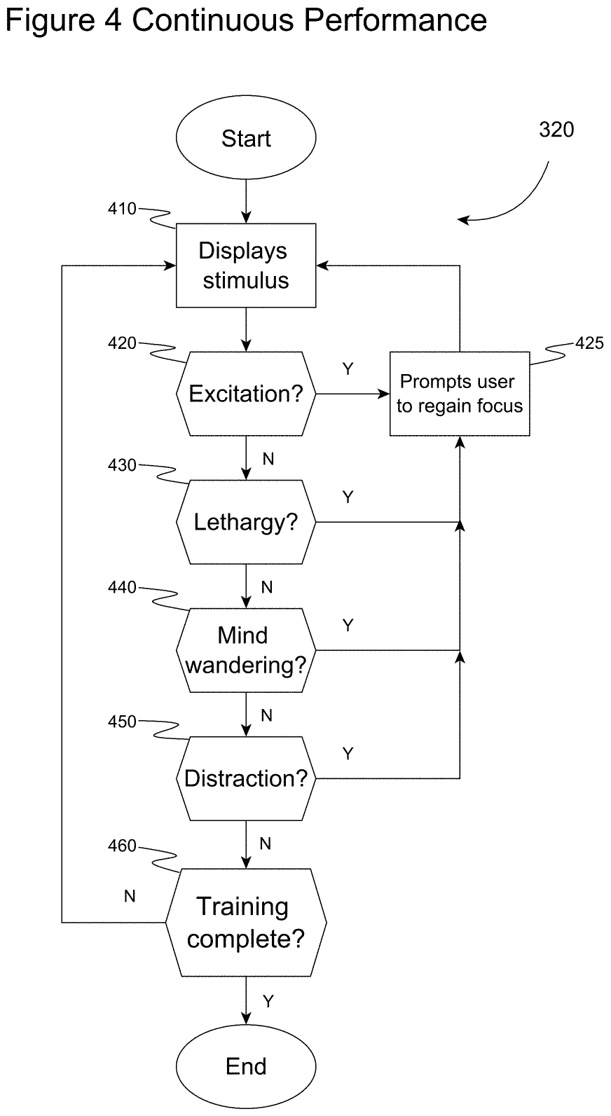

FIG. 4 is a block diagram specifically describing the continuous performance task 320. Each continuous performance task occurs in the same manners. First, the cognitive task displays a stimulus 410. The stimuli displayed 410 can occur according to, but is not limited to three specific manners: 1) inhibition of response, 2) low variance of response time, or 3) timing specificity. Then the user responds or does not respond according to the prescribed directions or based upon the type of cognitive task 310 chosen. No response by the user could be due to several situations. The first is as previously described; the stimulus does not require a response as per the prescribed directions. On the other hand, no response could be due to a failure to respond because of various uncontrolled behaviors such as excitation 420, lethargy 430, mind wandering 440, and distraction 450. While FIG. 4 has these four states (excitation 420, lethargy 430, mind wandering 440, and distraction 450) listed in this particular order, no specific order is required in the continuous performance task 320. Excitation 420 occurs when the focus of attention drifts away from the cognitive task to successive specific thoughts or external stimuli, such that the user's attention is continuously changing from one thought to another. Lethargy 430 is a state of mind that includes sluggishness, inactivity and/or apathy. Lethargy 430 afflicts individuals when the activity they should be doing becomes boring and repetitive. Mind wandering 440 is where the focus of attention drifts away from the cognitive task to another specific set of thoughts or set of externalities. Distractions 450 are external stimuli that cause the user to lose focus on the continuous performance task. More specifically, distractions are the unintentional movement of attention away from the intended focus that is caused by task irrelevant thoughts (internal distracters) or environmental events (external distracters). During the continuous performance task 320, the training task may prompt the user to regain focus 425 because the user has failed to respond to multiple stimuli. The cognitive task can repeat displaying stimuli 460 until the prescribed time period has occurred; however, the continuous performance tasks are not limited to the prescribed times. The continuous performance tasks may conclude after a predetermined number of correct and/or incorrect responses.

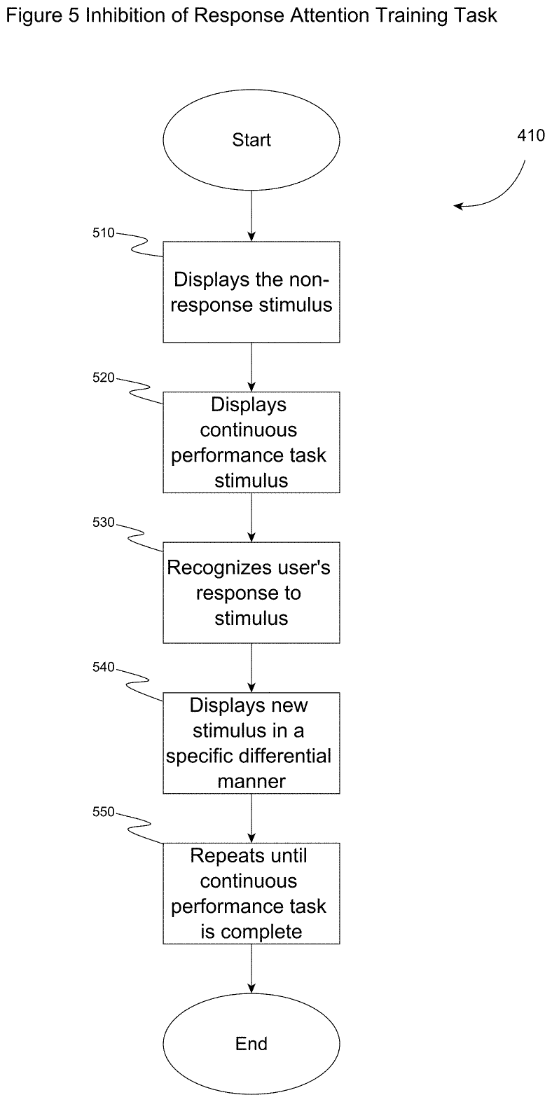

FIG. 5 is a block diagram describing one version of 410 according to the inhibition of response cognitive task. This specific cognitive task requires the user to respond to stimuli in a specific differential manner infrequently. Inhibition of response cognitive task indicates how well a user can control his/her attention. The cognitive task occurs in the following manner: 1) the non-responsive stimulus 510 is displayed, 2) the training task begins with a stimulus 520, 3) the user recognizes whether the stimulus requires a response, responds accordingly, and that response is recorded 530, 4) a new stimulus is displayed in a specific differential manner 540, and 5) stimuli are continuously displayed until the performance task is complete 550.

The following example describes the inhibition of response cognitive task. The training system displays a number between 0-9, which is the non-response stimulus 510. The training task begins and a number between 0-9 is displayed 520. The user recognizes the number as being either the non-response stimulus or not. The training system can recognize whether the user responds to the stimulus, e.g. touches the screen or does not touch the screen 530. After the response the training system can show a new number between 0-9 540. Once again, the user responds according to whether the number displayed is the non-responsive stimulus or not. The training continues for a prescribed amount of time or until the user has a prescribed number of incorrect responses 550.

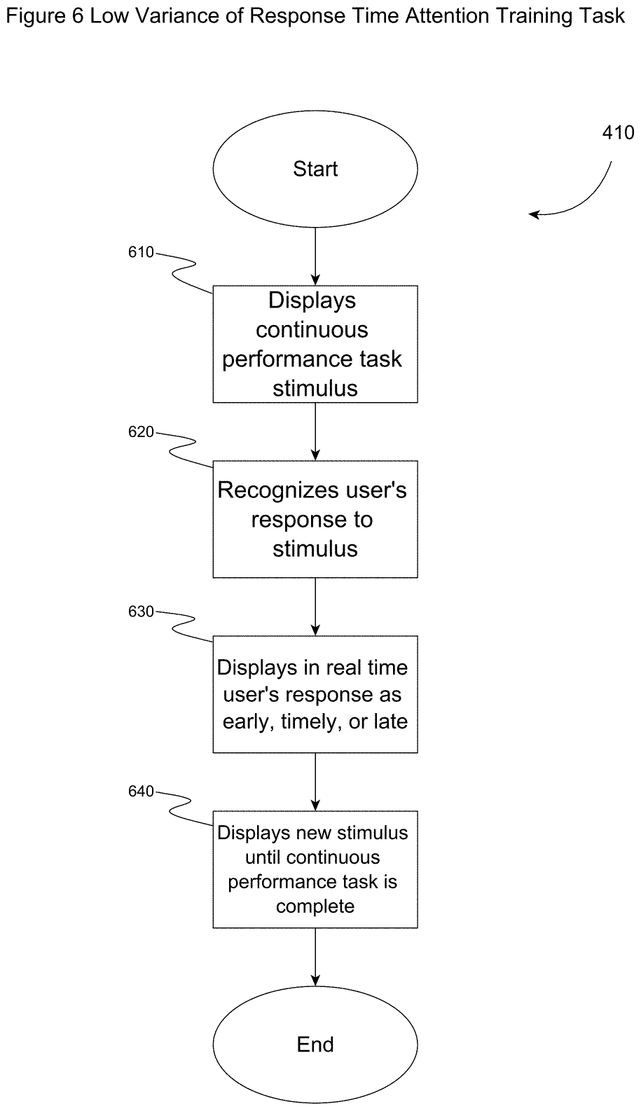

FIG. 6 is a second block diagram version of 410 describing the timing specificity cognitive task. The ability of an individual to specifically modify their response time on demand indicates superior control of the Meta-attention. The timing specificity cognitive task occurs in the following manner: 1) a stimulus is displayed 610, 2) a response is recorded 620, 3) in real-time, the training system displays the response as early, timely, or late 630, and 4) the cognitive task continues for the prescribed amount of time 640.

The following example describes the timing specificity cognitive task. An image of a firefly enters from any side of the display screen 610. The user focuses on the firefly and touches the screen as it passes through a specific, highlighted spot on the display screen 620. In real-time the training system can optionally inform the user as to whether the user's response was timely, early, or late 630. The training system can display a new firefly and repeat until the cognitive task is complete 640. Throughout the cognitive task, the firefly can flash on and off, requiring the user to maintain a consistency of response, whether the stimulus is visible or not, and determine when to touch the screen.

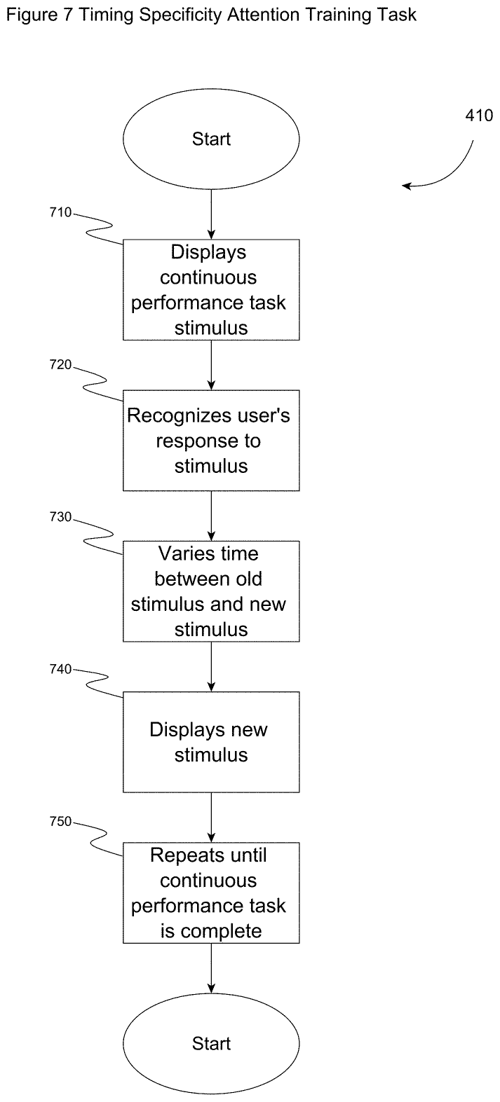

FIG. 7 is a third block diagram version of 410 describing the low variance of response time cognitive task. Low variance of response time reveals consistency of focus, which is a critical factor that indicates attentional control. The low variance of response time cognitive task occurs in the following manner: 1) a stimulus is displayed 710, 2) a response is recorded 720, 3) the timing between the new stimulus and old stimulus varies according to preset parameters 730, 4) a new stimulus is displayed 740, and 5) the cognitive task is repeated for a prescribed period of time 750. One embodiment of the timing specificity cognitive task is one that utilizes a specific detection task to capture the attention of the user. That cognitive task presents a visual Gabor patch on a visual background where the Gabor patch intensity is at or near the user's perceptual threshold. Perceptual threshold is set at the point where the presented stimuli are slightly above the intensity that would cause the user to not perceive them. Consequently, the user is required to determine if he/she has seen a stimulus and respond accordingly.

The following example describes the low variance of response time cognitive task. The display screen has a ripple, concentric circle like pattern with slight variation in color from the peaks to the troughs of the waves. The cognitive task displays a stimulus which is a patch of light, also known as a Gabor patch, which is slightly brighter than the background 710. This type of stimulus is called a near-threshold stimulus. The near-threshold stimulus is slightly above a person's perceptual threshold or the level of stimulus salience that is just enough to be perceived. The user taps anywhere on the screen after the stimulus is displayed and before the next stimulus. The system records whether or not the user responded 720. The cognitive task varies the timing between the old stimulus and the new stimulus 730. A new stimulus is displayed 740 and the cognitive task repeats for three minutes or until the user makes a total of three misses (e.g. a miss is defined as when the user does not respond after a stimulus has occurred within a pre-defined window of time following the stimulus) 750.

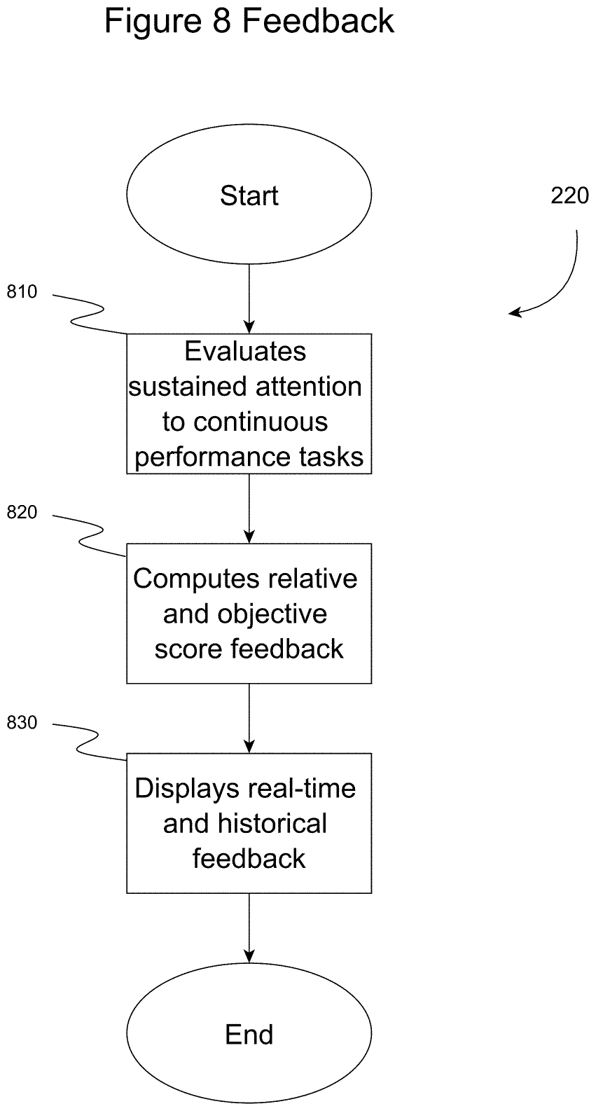

FIG. 8 is a block diagram describing the second step of a system for attaining Meta-attention 200, and the feedback mechanism 220. The feedback is important because it provides information to the user so that the user may better understand one's attentional abilities including attentional control as compared to oneself and compared to all those that have participated in the cognitive tasks. The feedback mechanism 220 occurs in a three-step process. The first step requires evaluating the responses from the different cognitive tasks 810. Second, the training system computes the relative and objective scores (longitudinal and normative) 820. Third, the training system displays any real-time and historical feedback 830.

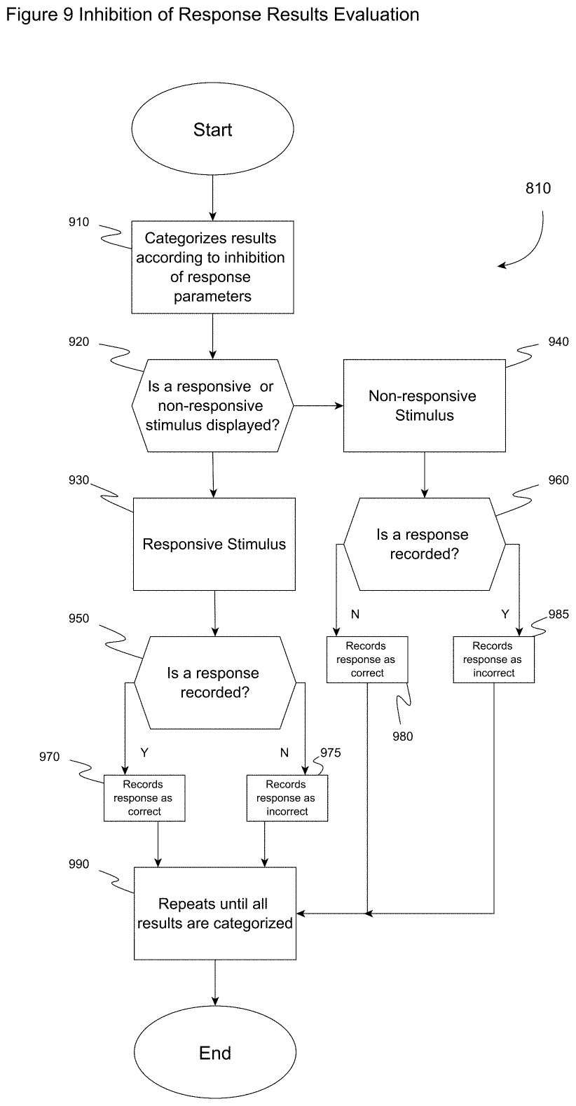

FIG. 9 is a block diagram of the feedback evaluation 810 describing the inhibition of response results evaluation. The training system categorizes each response according to the stimulus previously displayed 910. For each inhibition of response stimulus, the training system determines whether a responsive or non-responsive stimulus was displayed 920. If a responsive stimulus was displayed 930, the training system determines if a response was recorded 950. If a response was recorded, then the training system marks that response as correct 970 and repeat evaluating the remainder of responses 990. If the training system did not record a response and a responsive stimulus were displayed 930, then the training system marks that response as incorrect 975. Once again, the training system continues evaluating the remainder of the responses till all responses are categorized 990. If the training system displays a non-responsive stimulus 940, the training system determines if a response was recorded 960. If a response was recorded then the training system marks that response as incorrect 980 and repeat evaluating the remainder of responses 990. If the training system did not record a response and a non-responsive stimulus was displayed 940, then the training system marks that response as correct 985. Once again, the training system continues evaluating the remainder of the responses till all responses are categorized 990.

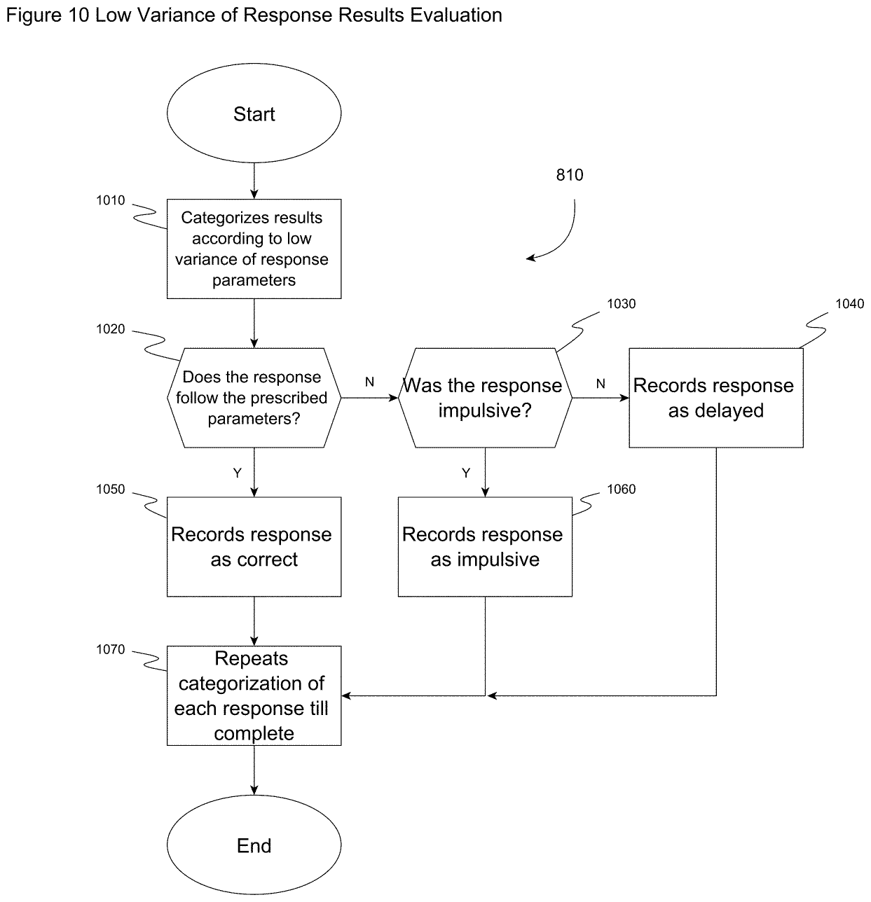

FIG. 10 is a block diagram of the feedback evaluation 810 describing the low variance of response results evaluation. The training system categorizes each response according to the low variance response parameters 1010. For each low variance of response recorded, the training system determines if the response recorded followed the prescribed parameters 1020. If the response recorded follows the low variance of response parameters, then the training system marks the response as correct 1050 and the training system continues evaluating the remainder of the responses till all responses are categorized 1070. If the recorded response was not according to the low variance of response parameters, then the training system determines if the response was impulsive 1030 or delayed 1040. An impulsive response is a behavior in which the user does not have the forethought as to the consequences of one's actions, acting on the spur of the moment. If the response was before the low variance of response parameters, then the training system records the response as impulsive 1060. The training system continues evaluating the remainder of the responses till all responses are categorized 1070. If the response was after the low variance of response parameters, then the training system records the response as delayed 1040. The training system continues evaluating the remainder of responses till all the responses are categorized 1070.

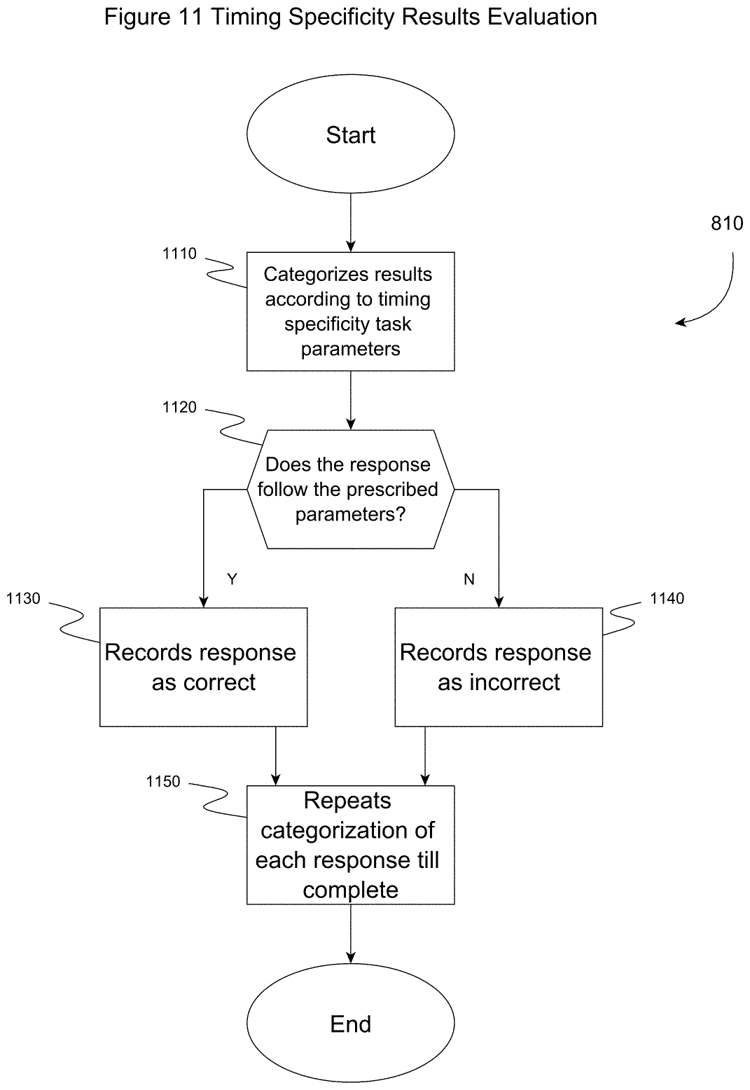

FIG. 11 is a block diagram of the feedback evaluation 810 describing the timing specificity results evaluation. The training system categorizes each response according to the timing of specificity task parameters 1110. The training system determines whether or not a stimulus was displayed 1120. If a stimulus was displayed and the user responded, then the training system records the response as correct 1130. The training system continues evaluating the remainder of the responses till all the responses are categorized 1150. If a stimulus was displayed and the user did not response, then the training system records the response as incorrect 1140. The training system continues evaluating the remainder of the responses till all the responses are categorized 1150. On the other hand, if no stimulus was displayed and the user responded to what he/she perceived as a stimulus, then the networks system records the response as incorrect 1140. This occurs because the user did not follow the prescribed timing specificity parameters. The training system continues evaluating the remainder of the responses till all the responses are categorized 1150

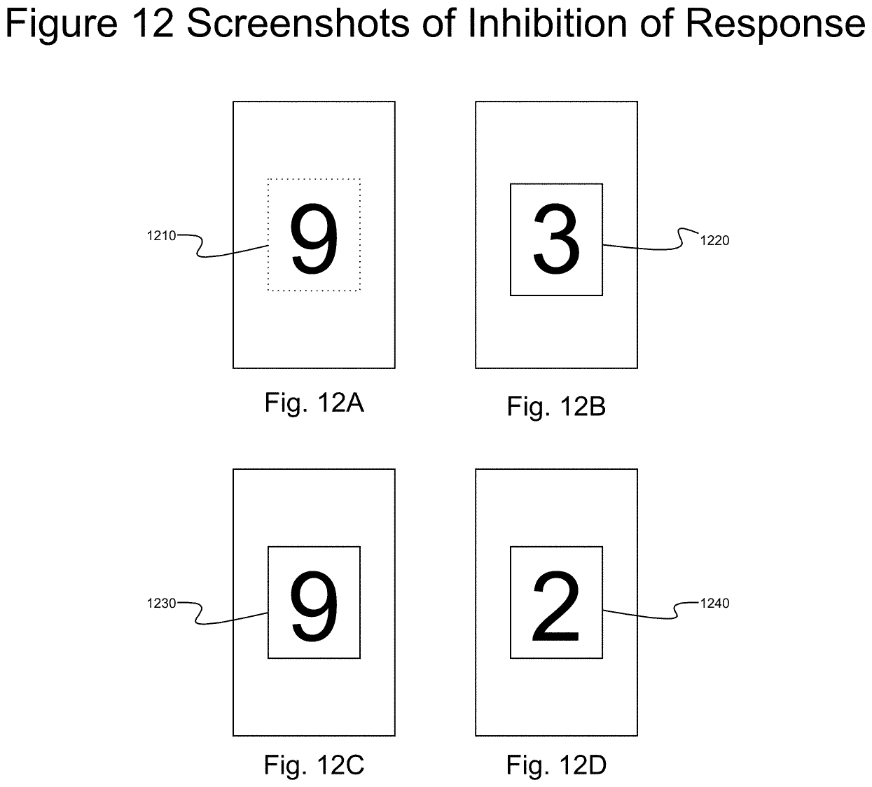

FIG. 12 is a collection of screenshots of the inhibition of response cognitive task. In FIG. 12A, the user is presented with the non-response stimulus 1210 with the dotted box designating that the number 9 is the non-response stimulus. Attentional control training commences after the user is presented with the non-response stimulus 1210. In FIG. 12B, the user is presented with a stimulus 1220. In this particular screenshot FIG. 12B, the user is presented with a stimulus 1220 which requires the user to recognize the stimulus as requiring action and tap the screen. The training system then displays a new stimulus as shown in FIG. 12C. In this particular screenshot FIG. 12C, the user is presented with the non-response stimulus 1230 which requires the user to recognize the stimulus as not requiring action and not tap the screen. FIG. 12D completes the collection of screenshots. In this particular screenshot FIG. 12B, the training system again displays a stimulus 1240 which requires the user to recognize the stimulus as requiring action and tap the screen.

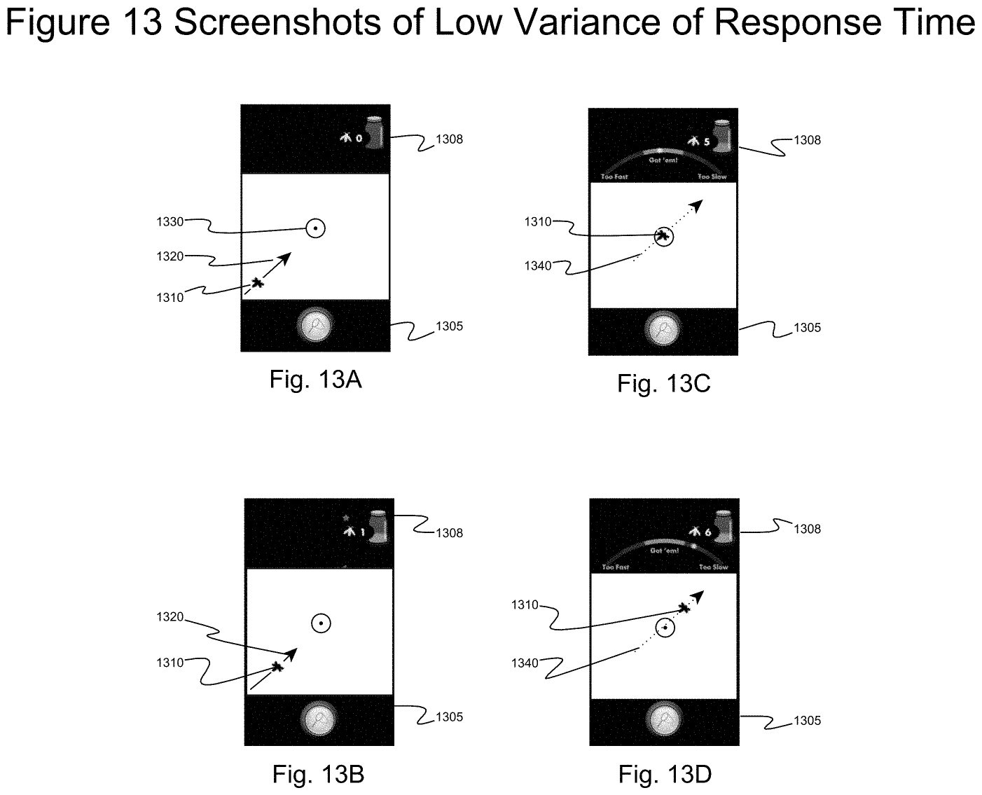

FIG. 13 is a collection of screenshots describing the low variance of response time training task. In FIG. 13A, the stimulus, or firefly, 1310 enters from the lower left corner of the screen. The arrow 1320 depicts the path of the firefly 1310 while the firefly is visible on the screen. Although no actual arrow 1320 is displayed during the cognitive task training, the firefly 1310 flies along a predetermined path 1320 until it reaches the target area 1330. When the firefly 1310 reaches the target area 1330, the user taps the net located in the lower area of the screen 1305. As the user correctly catches the fireflies 1310 in the target area 1330, the display at the top of the screen 1308 displays how many fireflies 1310 the user has correctly caught in the target area 1330. FIG. 13B shows the firefly 1310 further along the flight path as depicted by the arrow 1320. The display at the top of the screenshot 1308 shows that the user has correctly captured one firefly 1310. FIG. 13C depicts another aspect of the low variance of response training task. In this particular screenshot FIG. 13C the firefly 1310 had previously disappeared, meaning the stimulus was no longer visible, and was traveling along the path as depicted with a dotted arrow 1340. The user maintains attention on the cognitive task and determines when the firefly 1310 passes through the target area. The screenshot FIG. 13C further shows that the user correctly captured the firefly 1310. The meter located in the upper portion of the screen 1308 shows that the user correctly captured the firefly 1310 by displaying the white dot in the lighter green portion located above the “Got ‘em!” label. FIG. 13D is a screenshot of what happens when the user's response is too slow. The firefly 1310 was invisible, although visible in FIG. 13D for demonstration purposes only and not actually seen during training, and flying along a path as depicted by the dotted arrow 1340. The firefly 1310 is outside of the target area and has flown past the target area. The meter in the upper portion of the screen 1308 shows what happens when the user does not correctly capture the firefly 1310 and is too slow. The white dot is no longer in the lighter green section but has progressed into the dark green section and closer to the “Too slow” label.

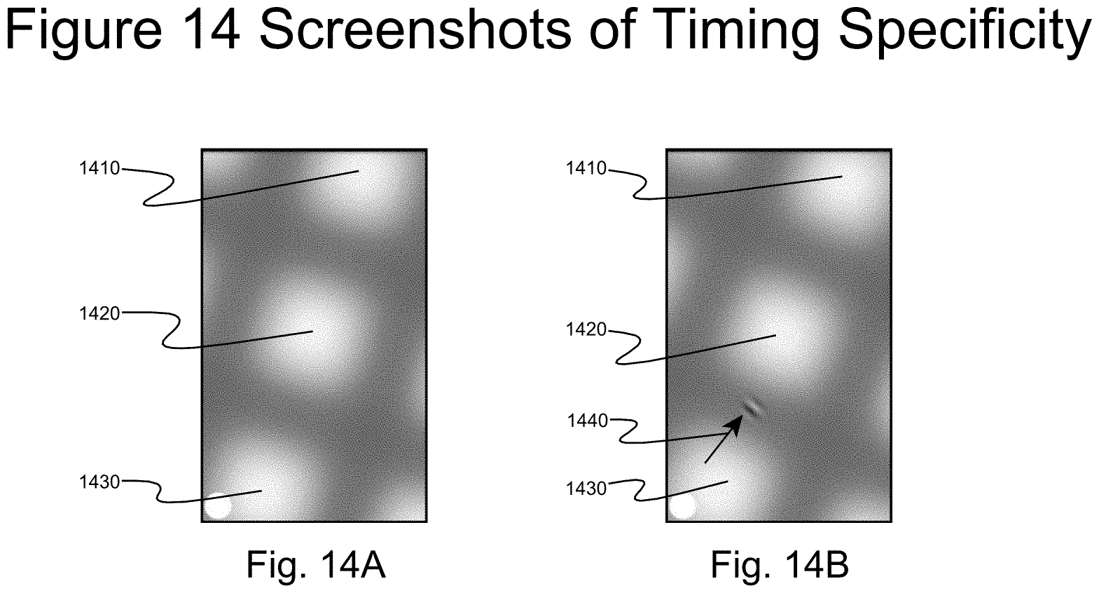

FIG. 14 is a collection of screenshots describing the timing specificity cognitive task. FIG. 14A shows the screen of the training task before any stimulus is displayed. The display screen is a contrast between the blue background and the diffuse white dots 1410, 1420, and 1430. The pattern created by the diffuse white dots 1410, 1420, and 1430 and the blue background produce less eye strain when viewing for extended period of time. FIG. 14B depicts what the screen looks like when a near-threshold stimulus, or Gabor patch, is displayed. The arrow 1440 is pointing directly at the near-threshold stimulus, or Gabor patch. When the user perceives a near-threshold stimulus 1440, the user registers the near-threshold stimulus 1440 and taps the screen.

It has been shown that patients (elderly with cognitive decline) can be differentiated on the basis of fMRI-based brain measures of distractor suppression versus target enhancement, demonstrating that these separate top-down control systems can be altered independently as well. This implies a need for intra-individual measures of brain processes over time to characterize maintenance of both processes: attending targets and ignoring distractors. Objective assessment tests must obtain brain measures of both components of attentional control and their intra-individual fluctuations during sustained attention. Detecting fluctuations over time requires continuous measures of both target and distractor processing. Doing so improves diagnostics and monitoring of the brain effects of treatment.

Neurophysiological Attention Test (NAT) utilizes a novel EEG method to measure infra-slow fluctuations in BOTH attending and ignoring simultaneously during sustained attention tasks. The NAT is the first EEG-based method for continuously tracking neurophysiological indices of attending targets and ignoring distractors simultaneously during sustained tasks. We adapt the steady-state visual evoked potential (SSVEP) method to take advantage of the fact that the magnitude of stimulus processing in sensory cortex (measured by the SSVEP) provides an index of attentional top-down control from frontal-parietal systems. Our method makes it possible for the first time to continuously measure the intra-individual variability (infra-slow fluctuations) of electrophysiological brain activity representing the top-down controlled processing of BOTH attended targets and of ignored distractors in ADHD. Measurements of infra-slow fluctuations can also be fMRI-based.

In some embodiments, However, it is challenging to define EEG measures that can be used unambiguously to measure the responses to each of multiple stimuli presented simultaneously. The SSVEP frequency tagging method allows the attended target signal and the ignored distractor signal to be identified by the frequency of the SSVEP. Each stimulus type (target, distractor) is assigned a flicker frequency (e.g., 15, 17 Hz respectively) that drives visual sensory cortices at the flicker frequency of each stimulus, thereby isolating and stabilizing the EEG activity corresponding to each stimulus type even when they are presented at the same time or even in the same location, e.g. in figure/background configuration. It is the infra-slow fluctuations in attending and ignoring that one can be aware of (meta-attention), and thus these measures can be used for assessment of meta-attention and for feedback about attending and ignoring and training meta-attention to improve attentional control. Many patients, for example those with ADHD, stress, anxiety and depression, have less meta-attention, e.g. they are less self-aware of their attention, or are less frequently self-aware of their attention. This results in negative symptoms and decrement of quality of life. Improving their meta-attention and thereby also their attentional control can greatly benefit these patients.

In accordance to the embodiments of the present invention, in addition to relating physiological indices to performance, the physiological indices themselves have functional significance. In fact, they can uncover important brain function differences, in the presence of similar behavioral measures, that can be differentially targeted by therapeutics. At a minimum, they can provide additional, otherwise unavailable, information to behavioral measures that may not completely differentiate sub-groups of patients (but suggest that there may be sub-groups), to aid in that differentiation.

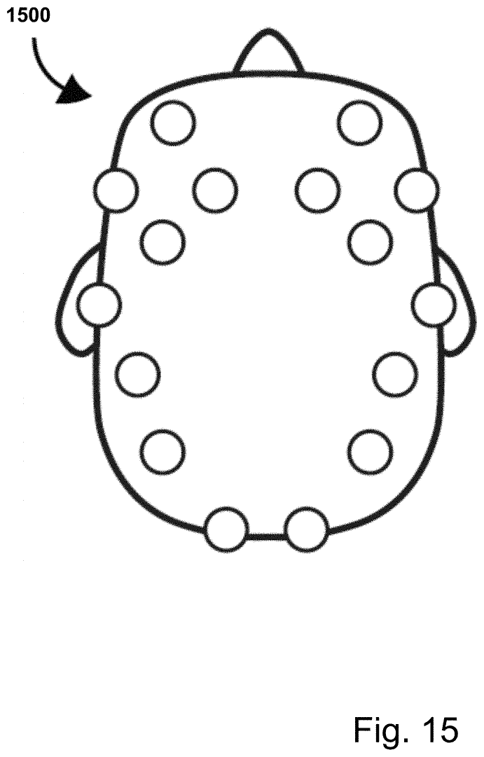

FIG. 15 presents an illustration of an example EEG sensor array, shown generally at 1500, that allows for measuring brain activity of a user of the proposed system. While not needed in some substantiations of embodiments of the present mental training systems, the added data collected by the EEG sensors may be beneficial to the AI analysis of the tasks presented, in order to determine guidance best suited for the user.

FIGS. 16A to 16C provide simulated screenshots of where an output device presents and instructs the user to attend to a first visual and/or audial (also referred to as “auditory”) stimulus, such as a visual circle (also referred to as “circular patch”) alternating between lighter circle 1620 and darker circle 1625, and flickering at a first frequency, generally approximately between 3 Hertz and 40 Hertz, and preferably substantially between 15 Hertz and 18 Hertz.

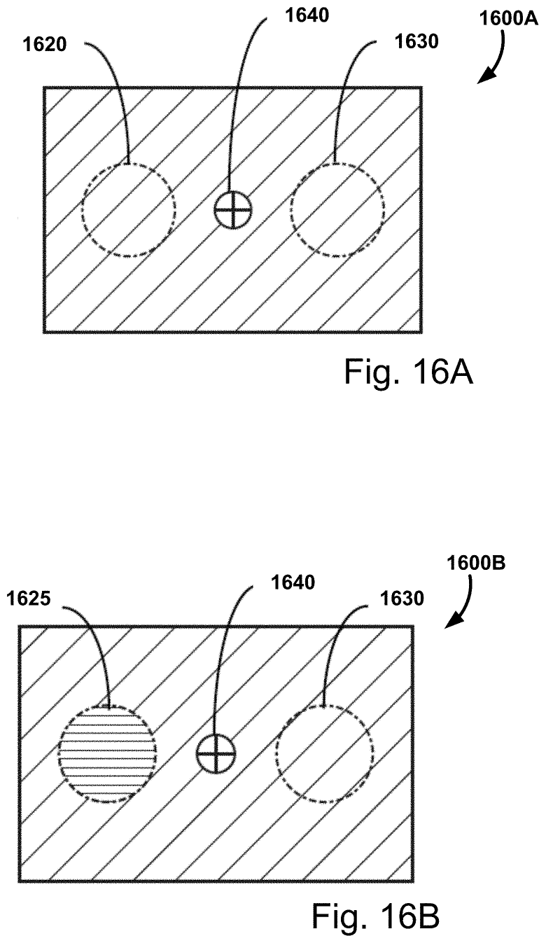

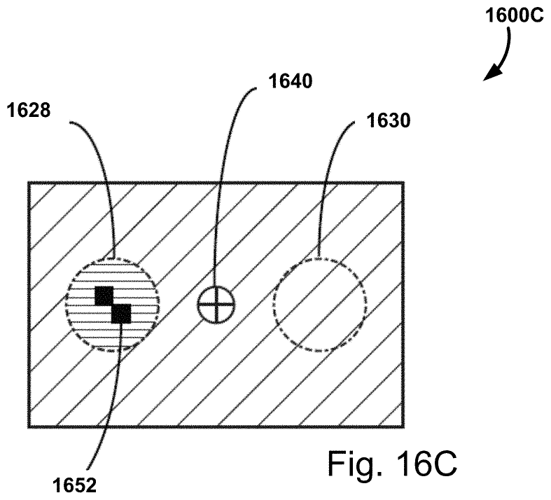

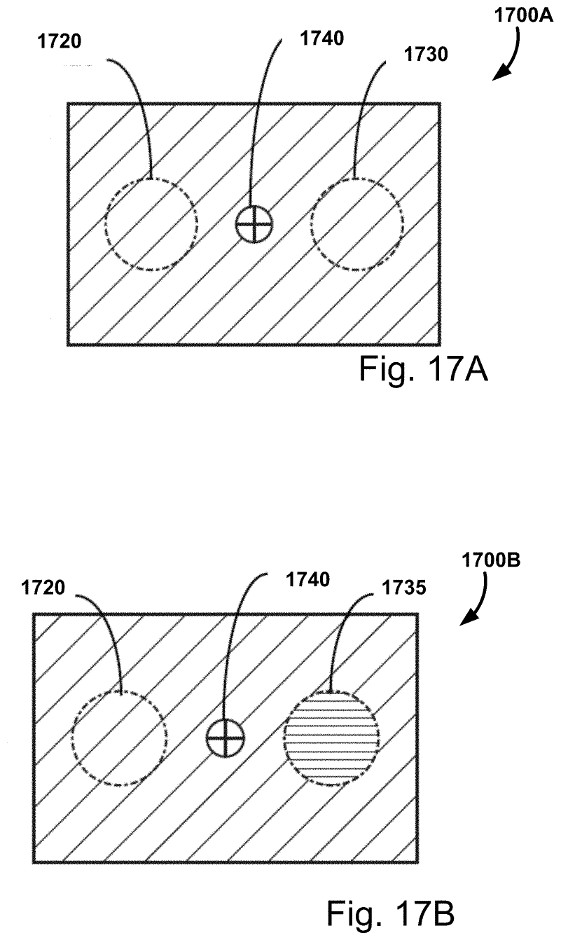

Referring also to the screenshots of FIGS. 17A and 17B, the output device also presents and instructs the user to ignore a second visual and/or audial stimulus via output device, such as another flickering visual circle alternating between lighter circle 1730 and darker circle 1735, and flickering at a second frequency, generally approximately between 3 Hertz and 40 Hertz, and preferably substantially between 15 Hertz and 18 Hertz.

The user may also be provided with an optional suitable focal cue, such as to focus user's eyes on fixation point 1640. In this example, the first flickering frequency can be 12 Hertz and the second flickering frequency can be 16 Hertz, and the fixation point 1640 can be located approximately midway between the attended circle 1620 and the ignored circle 1630. In some embodiments as illustrated by the respective screenshot 16C and screenshot 17C, a randomized plurality of targets and/or distractors, e.g. target 1652 and/or distractor 1752, can be presented to user either in attended circle 1628 and/or ignored circle 1738. Presentation frequency of targets and/or optional distractors can be approximately between one and five seconds, and can be randomized with respect to presentation rate, location and/or duration. Although squares are used for targets/distractors in this embodiment, other shapes are also possible, e.g., circles, ovals, rectangles, polygons, triangles or any other regular or irregular shapes.

A neural scanner measures the user's SSVEP (steady state visual evoked potential) and/or SSAEP (steady state auditory evoked potential). Neural scanner can be an EEG headset as seen at FIG. 15. The user's mental capability is computed using the fluctuation and/or SSVEP/SSAEP using the above described protocol. The computed capability can be provided to the user and/or a human therapist for evaluation purposed, and hence can be used for development of a personalized treatment plan. Likewise the AI driven guidance and feedback system can consume the EEG results as input vectors in order to match the user's SSVEP and/or SSAEP to training sets of SSVEP and/or SSAEP to determine what guidance is best for the user.

This can be computed by frequency tagging, e.g., using a moving window fast Fourier (FFT) of the EEG to extract the magnitude (e.g., amplitude) of the SSVEP signal over time, thereby yielding a waveform that includes the infra-slow fluctuations of the SSVEP magnitude over a sustained period of time (e.g., from about five seconds to about two minutes, preferably approximately between 10 seconds and 100 seconds). Note that the “raw” SSVEP signal includes measurable representations of the above described flickering frequencies.

Different frequencies may be measured based upon the cognitive task being performed by the user. For example, when measuring attention, the FFT of the waveform can be used to compute the magnitude of the Infra-Slow Fluctuations (ISF) in an exemplary frequency band (such as 0.01-0.2 Hz). The infra—slow fluctuations of the extracted SSVEP can be used to compose an attention score which is useful for example in diagnosing ADHD. This technique results in an attention score that aids clinicians in their assessment of an individual's capacity to attend and ignore inputs, in simple form, for example:

A(sub ignore)=Power(infraslow band over time)

A(sub attend)=Power(infraslow band over time)

A(sub performance)=Power(infraslow band over time)

Wherein A is the attention score for each.

These techniques can be summarized by the exemplary equations:

A_{I}=P_{I}(t)_{ISF} EQUATION A

A_{A}=P_{A}(t)_{ISF} EQUATION B

A_{P}=P_{P}(t)_{ISF} EQUATION C

Wherein:

A=attention score (indicate sub-I=Ignore; sub-A=Attend; sub-P=Performance)

ISF=InfraSlow Fluctuations—very low frequencies, such as from 0.01-0.2 Hz.

P(t)=Power over time for the infraslow frequencies

NOTE: The magnitude of the power can be found using a FFT, wavelet or other type of transform.

Other modifications and additions are also possible. For example, the magnitude of the waxing and waning of attention reflected in the ISF magnitude can also vary over tens of minutes and hours of the day, or across days or longer periods which can be measured with the ISF magnitude at different periods.

Alternatively or in addition, a new measure that combines the ISF with other EEG measures can be created by relating the ISF to other EEG measures extracted at the same time. For example frontal lobe activity could be found that co-varies with the ISF.

Many other modifications and additions are also possible. For example, it may be possible to present targets substantially outside of the attended area, and identify the targets using a color, a shape or an alphanumeric character (or any suitable symbol such as an Arabic character or Chinese calligraphic symbol).

There are also alternate methods for explicitly and/or implicitly incorporating a target with the attended area, such as superimposing a “target” onto the attended area, by, for example, substantially lengthening/narrowing the duration of the flickering pulse and/or varying the color, shape and/or size of the attended area. There can be any number of attended and ignored areas which can be located anywhere, even overlapping.

Turning now from the data collection side of the systems, outputs provided to the user will now be discussed. In the initial iterations of the proposed systems and methods, the user may rely upon a smart device, such as a smartphone or tablet, which allows for display of visual cues, audio cues, and usually vibration or other physical sensations. In the future however, it is entirely possible that such devices will be able to provide olfactory cues, or other sensory outputs.

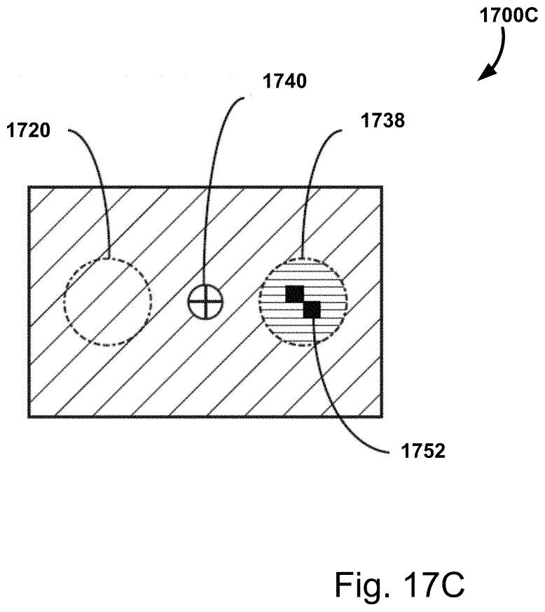

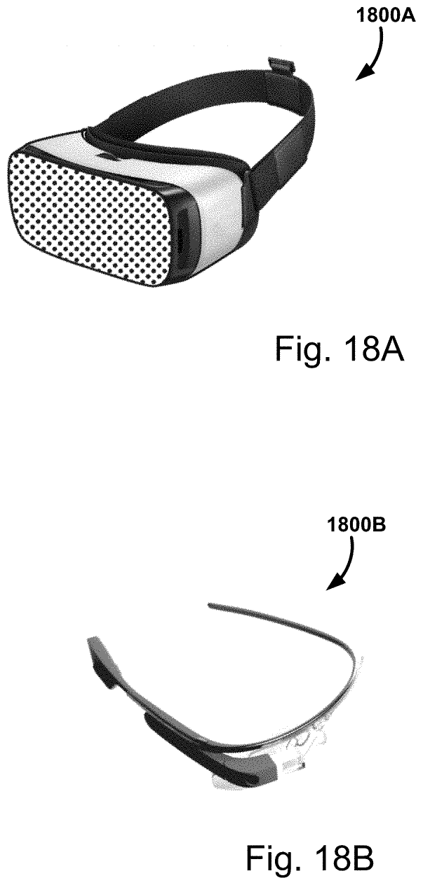

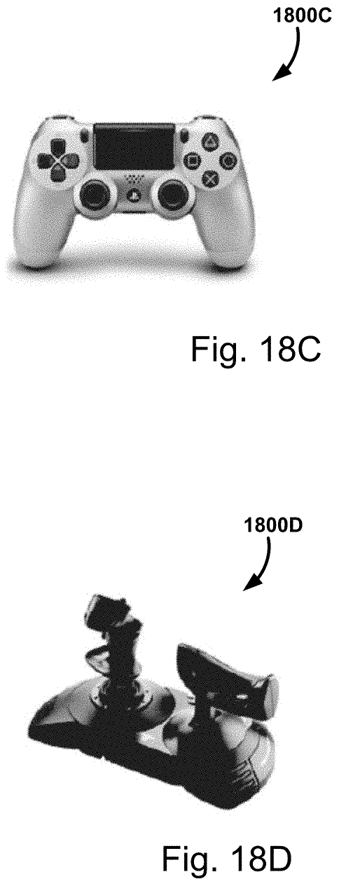

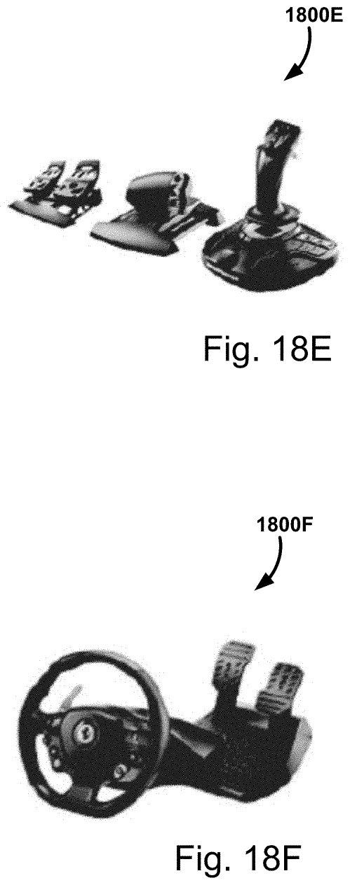

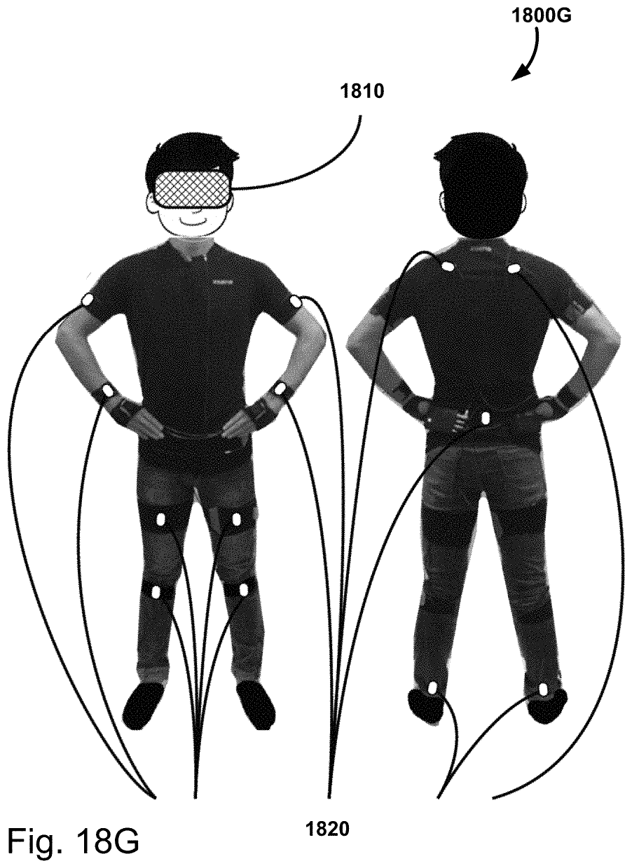

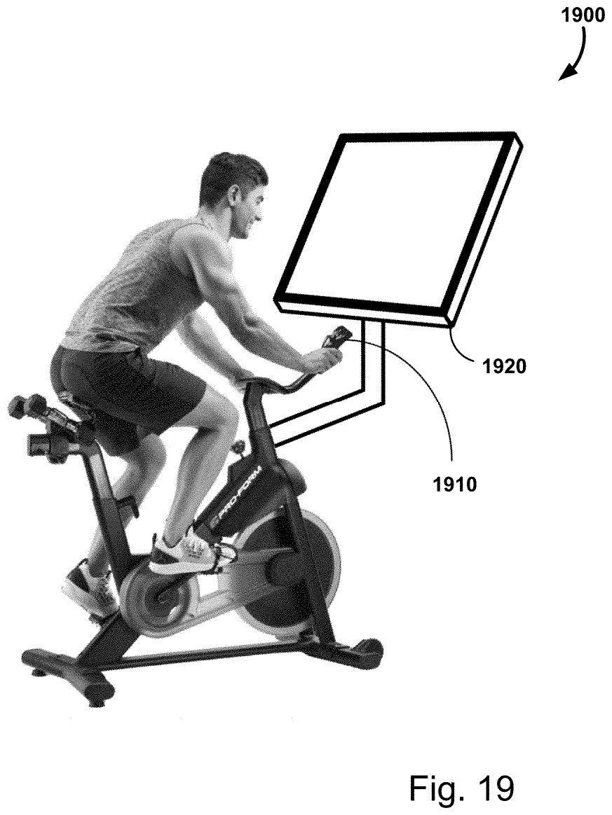

In other substantiations, other input and output devices may be leveraged by the proposed systems and methods. Examples of such IO devices are presented in reference to FIGS. 18A-18G. These may include VR headsets 1800A, augmented reality headsets 1800B, video game style controllers 1800C, joystick style devices 1800D, multi-limb IO devices 1800E, steering wheel style IO devices 1800F, and haptic sensors 1820 in conjunction with a headset 1810, as seen at 1800G. Other devices and environments that the disclosed systems and methods are particularly useful in are exercise settings, as seen in relation to an example scenario 1900 of FIG. 19. Here a user is actively engaged with a piece of exercise equipment 1910, which includes a screen 1920, or other IO device, such as the devices illustrated in relation to any of FIGS. 18A-18G. Other IO devices, such as holographic systems, are contemplated as being employed in embodiments. For the sake of brevity and clarity, however, these multitudes of additional devices are not explicitly shown. This in no way limits the scope of this disclosure, however.

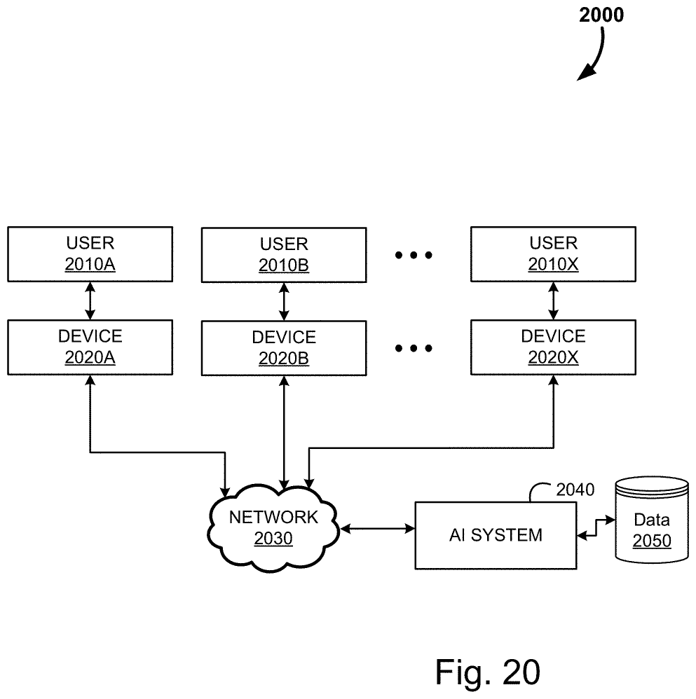

Moving on, FIG. 20 is an example of the cognitive measuring system, shown generally at 2000. In this example system a set of users 2010a-x, access the cognitive development system interface via a device 2020a-x. As noted above, these devices may include a large range of devices, and may even include an immersive environment comprised of many devices. For example, it is considered that a smart-home may be coupled to a device to control the entire environment around the user. The user may interact primarily through a smartphone or the like (as detailed extensively above), but additional contextual data may be collected from the larger environment (such as ambient temperature, noise levels, presence of others, etc.) as well as shaping the user's environment (such as altering lighting intensity and hue, playing particular music or other background noise, altering the thermostat settings, etc.). Said data regarding the devices and settings may be saved and later employed, even outside of the cognitive training setting, to help a user achieve a particular mental state. For example, settings around sleep training may be saved and employed at bedtime to help the users sleep, even when they are not actively participating in the sleep program.

Each user's devices are coupled to a backend AI system 2040 via a network 2030. In most cases the network is comprised of a cellular network and/or the internet. However, it is envisioned that the network includes any wide area network (WAN) architecture, including private WAN's, or private local area networks (LANs) in conjunction with private or public WANs. The AI system 2040 has access to data stores 2050 to effectuate the system's operations.

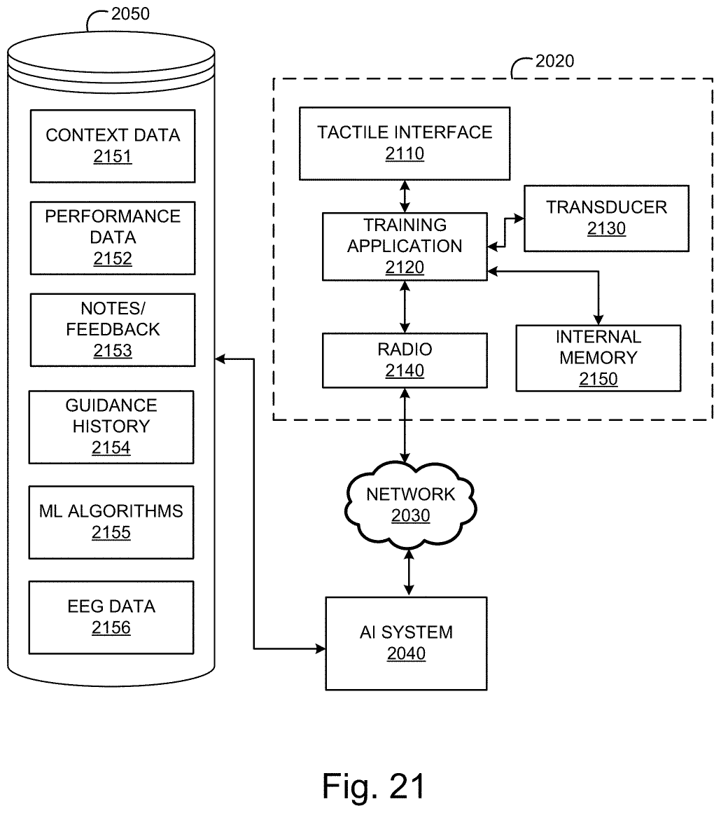

Turning to FIG. 21, a more detailed view of the user's device 2020 is provided. The user device generally includes a series of interfaces, including a tactile interface 2110, one or more transducers 2130 for generating and consuming audio signals, and a visual interface (not shown). Additional sensors and interfaces are likewise possible, including, for example, electrical/conductive sensors (to measure sweat levels and electrodermal responses in the user's skin), fingerprint sensors (to validate user identity), biometric sensors (to measure heartrate, temperature, respiration and the like), camera inputs that allow enhanced processing of user's eye dilation, facial recognition to determine emotion, eye tracking to measure attention focus, and the like. Additional sensors may be tethered to the device by wired or wireless connections. As noted before, as way of example, an EEG sensory array may be of particular utility in classifying the mental state of a user. Or a near-infrared spectroscopy array to classify mental state. Likewise, exercise equipment, haptic sensors, motion sensors, and the like may also be employed to track the user's physical state.

In addition to the larger data store 2050 leveraged by the external AI system 2040, the device may include its own internal memory 2150, which generally stores the training application 2120, which may be executed by the device's processor. A radio 2140 (or wired connection) couples the device to the network 2030, as previously discussed, and ultimately to the external AI system 2040.

In this disclosure, the local training application is differentiated as a different component from the backend AI system. This is a result of the AI processing requirements being substantial, and the local user's device is generally incapable of performing such operations. However, it is contemplated that, in the future, it is possible that local devices may have sufficient processing power and internal memory to effectuate local AI processing. In such situation, the AI system 2040 may physically reside within the user's device. Model updates will generally require connectivity to an external system that generates and updates the AI models, but actual model operation may be performed at the user's device.

The AI system accesses data located in the data store 2050 for training, modeling and selection of personalized guidance for the user. The data maintained in the datastore includes context data 2151 for each user. Context data can be extremely varied, and may include data about the user's psychological, physical and mental state, as well as information about the user's environment. Some of this data may be collected by the device and/or sensors, provided by the user, obtained from other users, or collected from ancillary sources (the weather, for example, where the user is located can be collected from the National Weather Service or similar third party sources).

Past performance data 2152 from prior sessions in the training tool for the user is also collected and stored, as is any notes and/or feedback 2153 supplied from the user to the system as part of an exercise. Historical guidance 2154 that was provided to the user is also stored. The trained machine learning algorithms 2155 are likewise retained in the datastore, along with EEG data 2156 and/or other massive data sets collected from the user (fMRI for example, in some embodiments).

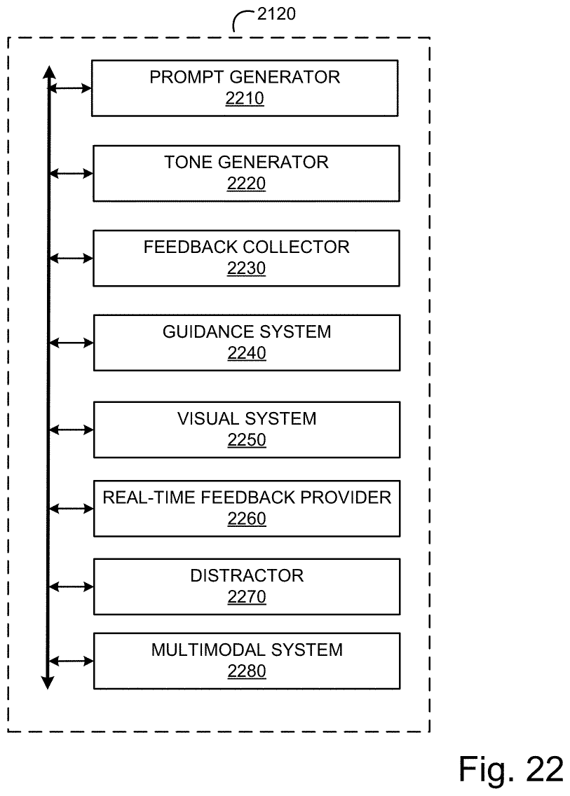

FIG. 22 provides a block diagram for an example of the training application 2120. In general, this system includes a prompt generator 2210 for guiding the user through mental exercises and evaluations. These prompts may be audible in nature and generated by a tone/audio generator 2220, visual in nature and thus generated by a visual system 2250, or of a differing output, such as olfactory, environmental conditioning, tactile or the like (produced by other output generators that are not shown for the sake of clarity and brevity).

Feedback from the user is elicited by a feedback collector 2230. The contextual data collected for the user, performance data (both real-time and historical) and collected feedback may be provided to the guidance system 2240 for generation of tailored guidance for the user. The guidance system may operate locally based upon a deterministic rule based model, and remotely, via the AI system to provide guidance based upon a more complex, context driven, data set that is consumed by the ML models for determining the most effective guidance. This guidance may be provided to a real-time feedback provider 2260 for relay to the user. A distractor 2270 may generate distractions and/or interruptions to the user, to assist in attention exercises, or to otherwise promote a changed mental state in the user. A multimodal system 2280 may be leveraged to present stimuli to the user in two (or more) mechanisms. For example, the given exercise may include both audio and visual stimuli that the user is expected to react to.

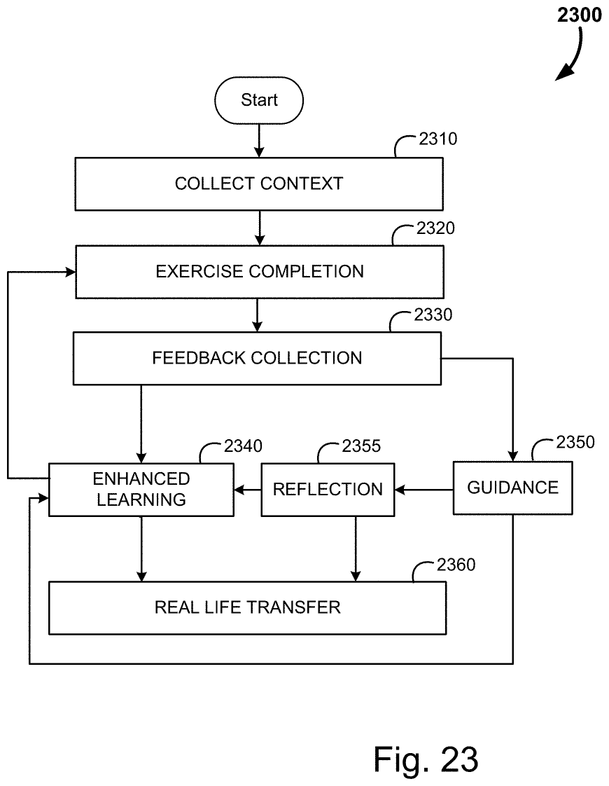

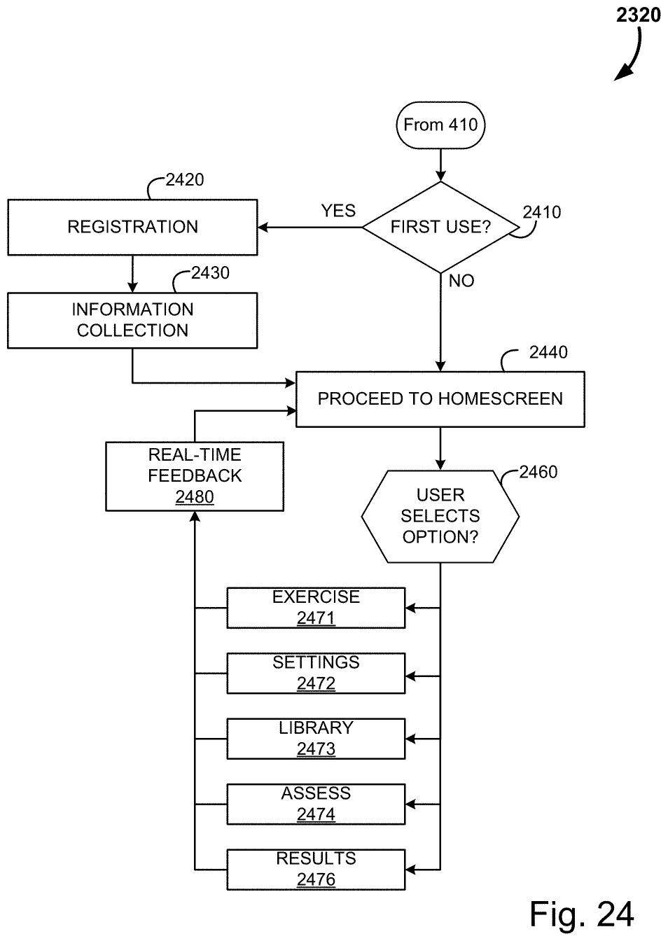

Turning now to FIG. 23, a flowchart of an example process for cognitive training is provided, shown generally at 2300. Initially, context for the user is collected (at 2310). Context, as previously touched upon, includes data provided by the user directly (such as a mental state and magnitude), environmental factors that can be collected, inferred or controlled, and biometric data that can be collected from the user (e.g., facial recognition for emotions, pupil dilation, skin conductivity, EEG feeds, etc.). After context is collected, the user is subjected to a mental exercise (at 2320). Exercise completion is provided in greater detail in FIG. 24. The first step is to determine if this is the first use of the system by the user (at 2410). If so, then the user is subjected to a registration process (at 2420) and basic information regarding the user is collected (at 2430). This includes basic bibliographical information, and generally an account set-up. After this the user is routed to a home screen (at 2440). The user is then provided a series of options, which the user is able to select between (at 2460). These options include performing an exercise (at 2471), adjusting the application settings (at 2472), accessing a library of guidance literature, manuals and other resources (at 2473), assessing their performance (at 2474) and accessing past results and performance (at 2476).

This listing is not limiting in scope, as other options are likewise available, such as journaling and guidance (not illustrated). Likewise, not all the options presented here necessarily are required to be present in the system. For example, “assessing their performance” may be omitted as a separate module, and instead be part of the loop in which the user performs the exercise, receives feedback, allows the user to reflect upon the feedback to gain insights and then replays the exercise in order to apply the insights. This reflection step is augmented by guidance (provided separately from the options) to enhance the generation of insights. The past performance data may likewise be leveraged in the formation of insights. Regardless of option selected, the user is then provided relevant real-time feedback based upon their selection (at 2480) and may then be returned to the home screen. Real-time feedback is provided during the exercise, whereas end of session (EOS) feedback may be provided after the exercise has been completed.

For the results process 2476, comprehensive data may be accessed, which includes: the Data/Results from the last session, also their entire dataset for all sessions and any other data input, parsed into modules that are meaningful to the user. Such as type of results (accuracy, RT variability, Self-reported improvement scores, etc.) and time (display data for a day, week, month etc.). Patterns in the performance and self-report data as well as relationships/patterns in those data are displayed together with all contextual data over time as determined by algorithms and AI. This is performed for different categories such as an association between improvement with one exercise type more than another, or improvement in that exercise as a function of time of day. These performance data and self-reports are related to the content in their notes and to their contextual data.

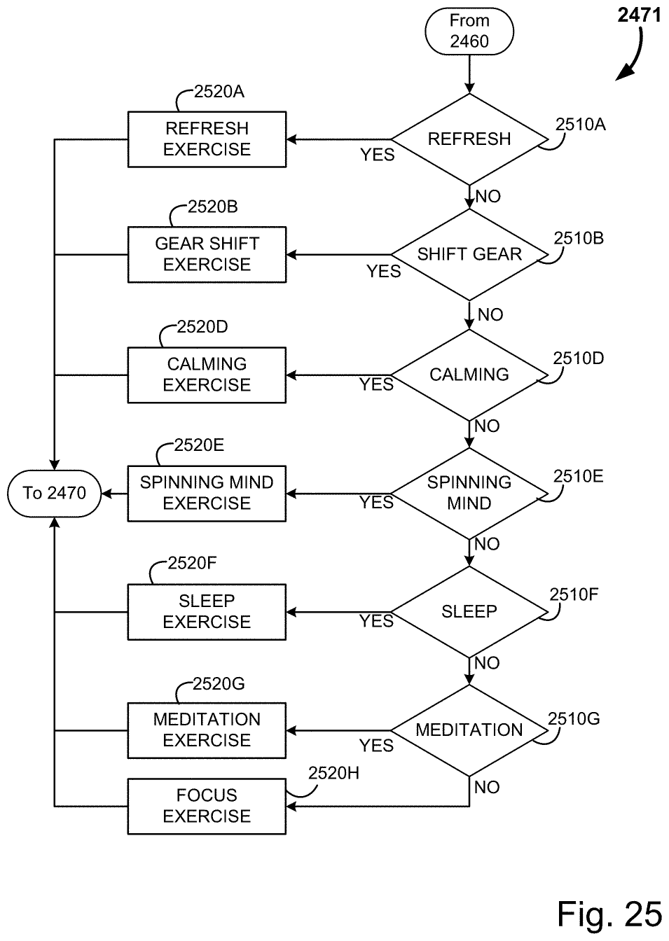

The exercise process 2471 is provided in greater detail in relation with FIG. 25. In this example process the user is presented a set of differing exercises, including but not limited to refresh, shifting gears, calming, spinning mind, sleep, meditation and focus. While these exercises will be discussed in considerable detail below, they are not intended to be limiting, and other exercises are entirely possible. For example, a time to recover from distraction exercise (including emotional/stress triggers) may also be included in the above listing, in some embodiments.

For ‘Focus’, warm energizing colors may be employed. The intention is to immerse the mind in background sounds, and return a wandering mind to the target. Some may recognize this as a “mindfulness” type meditation state. The user may have the ability to configure the sound scene, time and difficulty of the exercise. The timing of the exercise is typically 2 minutes, it is at a fast pace, with more lively audio scenes, and harder options, such as requiring responses every other target (tone). Assessments for this exercise could include accuracy, the variability in response speed over time—a measure of consistency in sustained attentional control. As with other exercises (except sleep), the assessment for the exercise is performed at the end of the exercise, and includes a request for feedback regarding improvement in the relevant mental state. These answers are tracked, using a Likert scale for example, to show the users relevant patterns in their data. For example, under this focus exercise, it may show that focus is improved more or less at particular times or particular days.

For ‘Shifting Gears’, the color scheme may be neutral and cool. The graphics are relaxing. The intention of this mode is to place attention gently on a target tone and allowing it to occupy the user's thoughts. It's also intended to clear the user's mind such that they are capable of preparing for a change in activities and to be able to be focused for that activity. The parameters that may be customized by the user are again the sounds available, time and difficulty. The target time for the exercise is 4 minutes, and it is a slow pace. Metrics for this exercise that are assessed include the variability in response speed over time—a measure of consistency in sustained attentional control.

For ‘Refresh’, again the color scheme may be neutral and cool. The graphics are relaxing. The intention of this mode is to address mental fatigue, to put thoughts and thinking on hold, to relax while giving just a little attention to the target sound in order to keep the mind from drifting too much. Again, parameters that may be customized by the user are again the sounds available, time and difficulty. The target time for the exercise is 4 minutes, and it is a slow pace. Metrics for this exercise that are assessed include the variability in response speed over time—a measure of consistency in sustained attentional control

For ‘Spinning Mind’, cool colors and calming graphics are employed. The intention of this exercise is to let the user's thoughts go, diminish recurrent thinking and attachment to the thoughts, and allow attention to be drawn away from the thoughts by attending to the target sound. Parameters that are configurable for this exercise include sounds and time. The target time for the exercise is 4 minutes, the pace is slow, and a breathing pre-exercise is required. Metrics for this exercise that are assessed include the variability in response speed over time—a measure of consistency in sustained attentional control

For ‘Calm’, a subdued color scheme with relaxing graphics are employed. The intention of this exercise is to let the user relax and reduce stress by periodically taking deeper breaths, opening up their mind and being immersed in the sounds while gently paying attention to the target sound to keep them focused at the same time. The target time for the exercise is 4 minutes, the pace is slow, and a breathing pre-exercise is required. Metrics for this exercise that are assessed include the variability in response speed over time—a measure of consistency in sustained attentional control.

For ‘Sleep’, a dark color scheme that avoids blue hues is used, with sleep graphics. The intention of this exercise is to allow the user to let go of thoughts in order to prepare the user for sleep. Only sound options are configurable by the user, while the exercise is on a preset timer. The exercise includes an active response segment that lasts for 4 minutes, which then transitions to an audio segment. There is no assessment with this exercise, the system merely times out.

For ‘Meditate’, a calm color scheme is utilized. The intention of this exercise is to allow the user to let go of their thoughts and feelings, immersed in the background sounds, while gently focusing on the target sound as an anchor for their attention to come back to if their mind drifts to thoughts or feelings. The configurable parameters include the option for a simple mindfulness preparation versus an extended meditation session. The target time for this exercise is 4 minutes (extendable), and has a slow pace. Metrics for this exercise that are assessed include the variability in response speed over time—a measure of consistency in sustained attentional control.

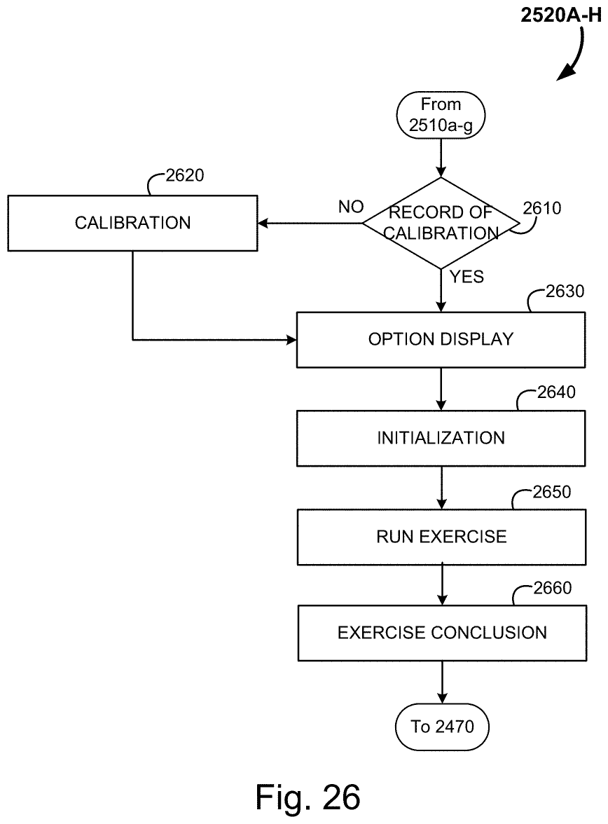

The user may select between the different exercises (at 2510A-G, respectively). Subsequently, the user is routed to perform the given exercise (at 2520A-H, respectively). FIG. 26 provides a genericized example process of the exercises being performed. Initially, there is a query if there is a record of calibration for the exercise (at 2610). If not, the system calibrates the user for the given task (at 2620). The calibration process involves playing a background sound scene continuously while playing the target sound, with the user required to tap the screen when they hear the target sound. The amplitude of the target sound is modulated and tapping feedback is collected until the desired perceptual level is reached. The user is then presented options (at 2630), and the program is initialized (at 2640).

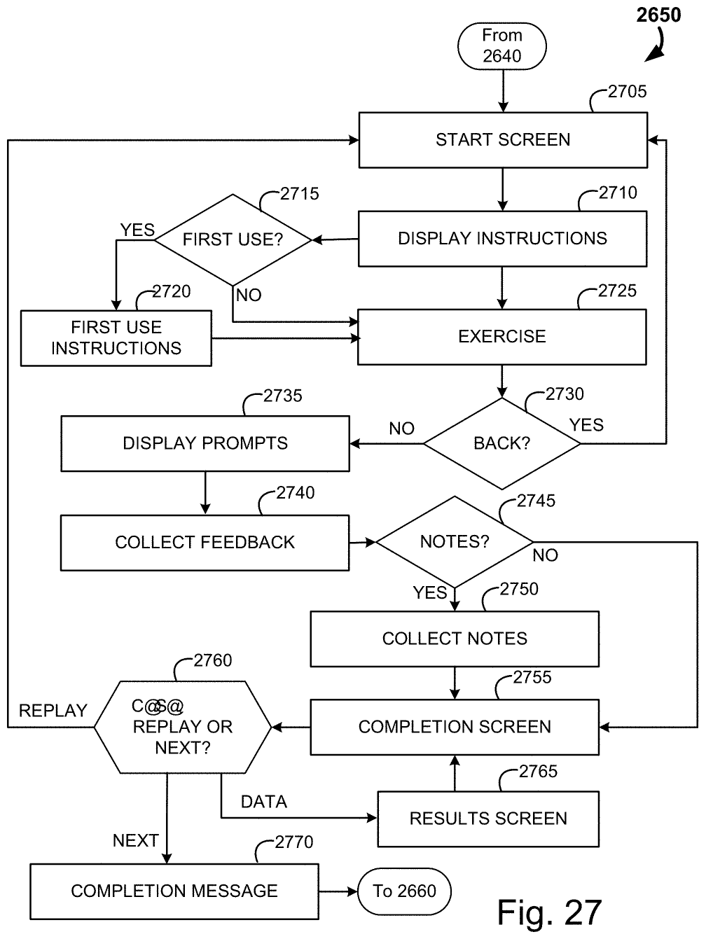

The exercise is then run (at 2650) based upon the type of exercise selected, as noted in the previous figure. The exercise is then concluded (at 2660). FIG. 27 provides a more detailed example of running the exercise. Initially the user is presented a start screen (at 2705). The instructions for the exercise are displayed or played as an audio or video file (at 2710). A determination is made (at 2715) if this is the first use of the program by the user. If so the user is additionally presented, or played, first use instructions (at 2720). The exercise, as previously discussed, is then initialized (at 2725).

After the exercise, the user is given an option to go back to the start screen, or to proceed further (at 2730). If the user proceeds, they are presented a prompt (at 2735). Again, the prompt may be visual in nature, or may include audio, tactile, olfactory, or other inputs (or a combination thereof). The user then reacts to these prompts, and this feedback by the user is collected by the system (at 2740). The user is also presented the option, at the end of the exercise, to provide notes (at 2745). If the user opts to provide these notes they are collected (at 2750). Notes may be processed by natural language processors to identify conceptual information. This may be performed by normalization of the text, parsing the text by constituent parts, and matching tokens in the parsed text to a conceptual lexigraphy hierarchy, based upon distance measurements between the token and the given abstracted concept. These concepts may be provided to the AI system as inputs to generate additional contextual information for the user.

Regardless if notes are collected or not, the user is then presented with a completion screen (at 2755). The completion screen can provide the user a congratulations message and options to either view their data from the exercise, replay the exercise, or complete the exercise (at 2760). If the user were to wish to replay the exercise, they are routed back to the start screen. If the user is interested in viewing her data, she would be routed to a results screen (at 2765) which displays the user's data measures for the given exercise and/or data for past exercises as well, in addition to data analytics derived from machine learning pattern recognition. Such as their performance data from the exercise (e.g. number of hits and misses) and derivative measures from those data (e.g. the variability in response time over the period of the exercise); the user's responses to the prompts/questions (e.g. are you more calm now) and the user's notes. In addition to display of the user data, the user may be requested to provide feedback and reflect upon their performance in a guidance framework in order to generate insights. This also allows the user to repeat the exercise, improving their performance on each iteration. After reviewing their data the user is asked to reflect upon their performance, and consider it together with what they are learning via guidance that is provided to them (not shown). This provides the user insights into how best to control their attention and how to improve on the next exercise. Subsequently, the user is re-routed back to the completion screen. Lastly, if the user selects to move on, the user is presented with a completion message (at 2770) and the exercise completes.

It should be noted that the above disclosed method of exercise operation is exemplary, and an attempt to genericize the process for the sake of clarity and brevity. In reality, each of the various exercises may deviate from the given process. For example, for the sleep exercise, after the system collects feedback on the user's progress, the system may slowly wind down. This may include playing soothing sounds/music, and a gradually lower amplitude, dimming the screen slowly, and requiring no further input or prompts to the user. In such an exercise, the user is not asked for notes, there is no completion screen, and the user does not select between options for the exercise to fully complete. As such, it is intended that the above exercise processes are merely illustrative in nature, and are in no way intended to limit the scope of the system's operation.

Returning now back to FIG. 23, after exercise completion (at 2320) additional feedback regarding the exercise itself, the provided notes and other contextual information is all consolidated (at 2330). The backend AI system leverages all of this information as inputs into the ML models to generate an output of guidance (at 2350). Guidance may include providing lessons to the user about the conceptual constructs for the many aspects of attention and attentional control, the relationships between attentional control and real-life contexts, and the benefits that attentional control provides to a person's well-being and performance in their everyday life; and/or lessons about learning-processes that facilitate faster and deeper learning of attentional control such as reflection, actively building new meaning, taking new actions to build attentional control and ways to actively transfer attentional control skills to everyday life.

In addition to providing guidance, the user naturally “gets better” at the mental tasks presented in the exercises. This occurs in two ways: due to practice and learning, and secondly due to insights gained during the reflection (2355) that occurs in the guidance process (2350). This enhancement of learning (at 2340) assists in improving performance in subsequent exercises. Likewise, this enhancement in the user's learning, coupled to the guidance provided, is applicable to real-life situations. This transfer of skills from the system, to the user's everyday life (at 2360) is the ultimate goal of the system.

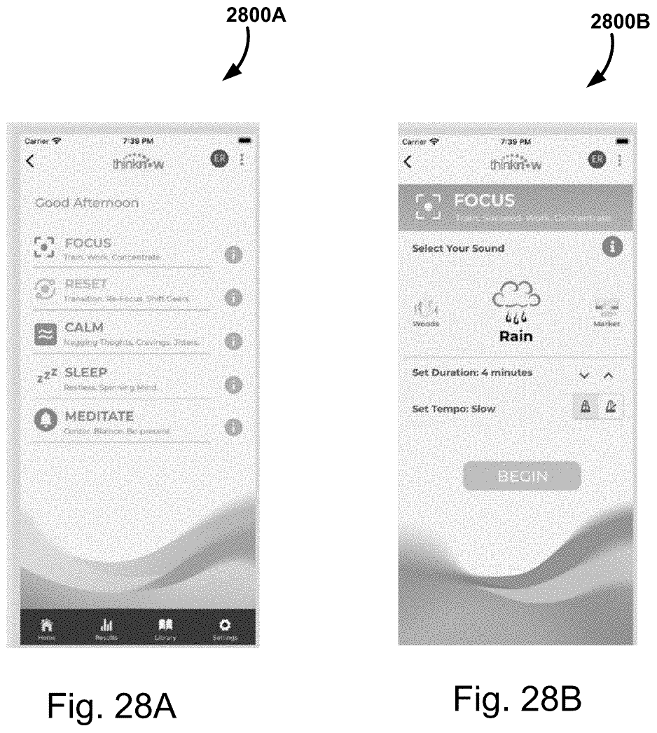

Turning now to FIGS. 28A and 28B, these illustrations provide example screenshots of the cognitive training, in accordance with some embodiments. In these figures, the display is primarily a visual display suited for a smartphone or similar device. As noted however, additional interfaces are common, including VR headsets, larger displays integrated into physical exercise equipment, and the like.

In FIG. 28A, the screenshot provided presents one example of a listing of mental exercises the user may select between, shown generally at 2800A. Compared to the listing of mental exercises disclosed previously, it should be noted that the display presents merely a subset of these mental exercises. This is for the sake of brevity, and this embodiment is but one substantiation of an aspect of the presently disclosed invention.

In this example interface, the user is able to return to the pervious welcome screen by selecting the ‘back’ button at the top left section of the screen. The user account and settings may be accessed by the set of ellipses at the top right side of the screen. Each of the exercises may be selected directly, or information for the given exercise may be accesses by selecting the “i” symbol next to the given exercise. Along the bottom of the screen, there are quick access icons to bring the user back to the home screen, view prior results, access their library of resources and guidance, and access their settings.

When a user selects an exercise, they may be taken to a start screen for the given exercise. An example of such a screen is provided at FIG. 28B, in relation to 2800B. This example is for the ‘focus’ exercise. The color scheme for the given exercise may be tailored to the exercise type. For example, soft warm colors promote sleep, whereas blue hued colors are most effective at blocking the secretion of melatonin. For this reason, the ‘sleep’ mental exercise may be audio entirely in nature, or when a visual component is desired, very low light, non-blue colors may be employed, by way of example.

Likewise, the set of sounds played, or selectable by the user, may be tailored based upon the exercise selected. For example, for the sleep exercise, very soothing and quiet sounds may be selected, whereas for focus, even but louder sounds may be provided for the user. Even the same types of sounds may differ between the mental exercises. Again, returning to our prior examples, rain in the sleep exercise may include a soft drizzle type sound, whereas a focus exercise rain sound may include thunder and a downpour of rain sounds.

As seen in this example figure, the user may select among a variety of suitable sound options for the mental exercise, and select a duration (within an accepted range for the given exercise). Likewise the tempo for the exercise can be selected by the user. Once all parameters are thus set, the user can begin the exercise.

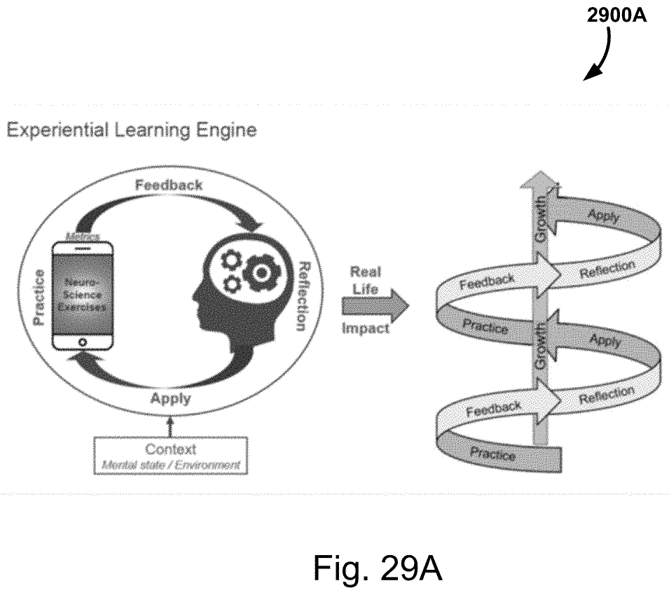

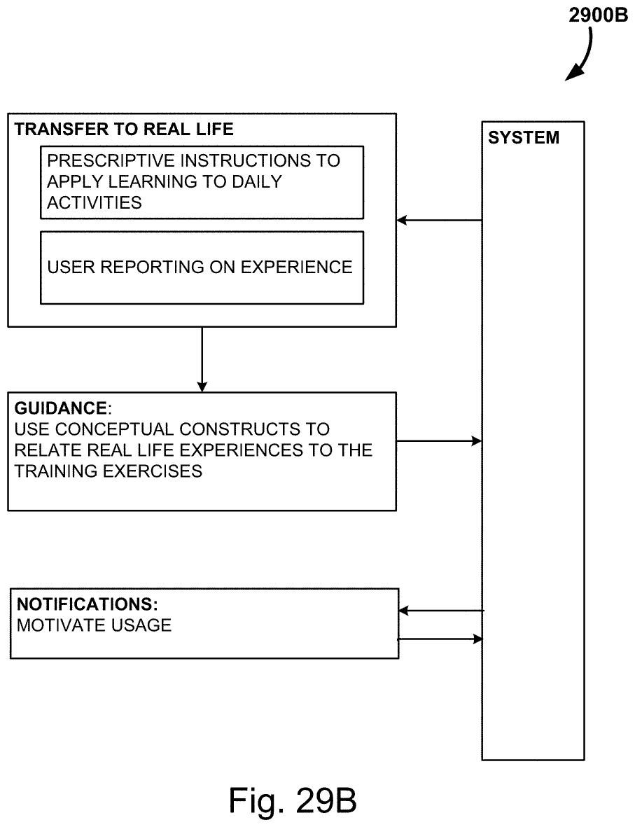

Turning now to FIG. 29A, an illustration 2900A of an example for the framework for improving cognitive functioning through attentional control and awareness through the disclosed experiential learning processes is provided. Again, here we see contextual information being fed into the system for consumption by the AI models. The user practices the exercises which generates feedback for the system in the form of metrics and notes. This information, in addition to the context information, may be leveraged locally using a rules based engine, or remotely using the AI modeling system, to generate feedback/guidance that is then supplied to the user. The user can reflect upon the feedback/guidance to deepen their learning, and ultimately apply the learnings to improve their performance on the exercises in future iterations. This cyclical loop of improvement is not a closed system. The learnings and improvements in the mental acuity of the individual is transferable to real life situations. This results in an iterative process that leads to personal growth, as represented in the illustration. FIG. 29B provides an illustration 2900B of the transfer of the learnings and skills gained by the system to real life situations. In this example illustration, the system provides the user with prescriptive instructions to apply the learnings. This, in combination with the user reporting on her experiences, helps to transfer the learnings, in a concrete manner, to real world scenarios. The reporting of experiences is stored in a database of the system which the AI can leverage to provide feedback to the user; effectively generating a feedback loop. In addition, the user is provided guidance in the form of conceptual constructs to relate real life experiences back to the training exercises.

At the same time, the system may provide the user with notifications to assist in motivating the user to continue engaging with the system. Effectively, this process of guidance and transfer of learning is a loop—the exercise teaches an aspect of attentional control, then the user implements that control in real life activity, then user sees how the control learned in the exercise impacts real life thereby making the learning in the exercise more relevant/meaningful and useful in improving real life.

In addition to the notifications, or possibly as part of the notification process, in some embodiments users may get credit for improving their cognitive control and well-being by doing other activities with other devices and apps. This is generally known as “gamification” of the process. Examples of this are receiving credits, badges, or other rewards for activities such as running, yoga, mindfulness class or other cognitive apps. The benefits of these other activities are revealed to the user by seeing how these activities impact their ability to control their attention during the present exercises, e.g., their scores in the above disclosed exercises. As a result the user gets cognitive control/well-being credits for improving their scores on the presently disclosed exercises after doing these other activities.

The factors determining which guidance to provide the user, and whether to move the user from a given module of an exercise to a more advanced module, is based upon several factors. These may include the number of sessions a user completes (in aggregate, or for a given type of exercise). The user's performance on the exercise may likewise help determine guidance and advancement in modules. The user's pattern in usage, context, user defined goals, and user desires/feedback may also all be leveraged by either the rule based engine, or by the AI models, to determine when the user is ready for advancement and/or the type of guidance the user should get as feedback.

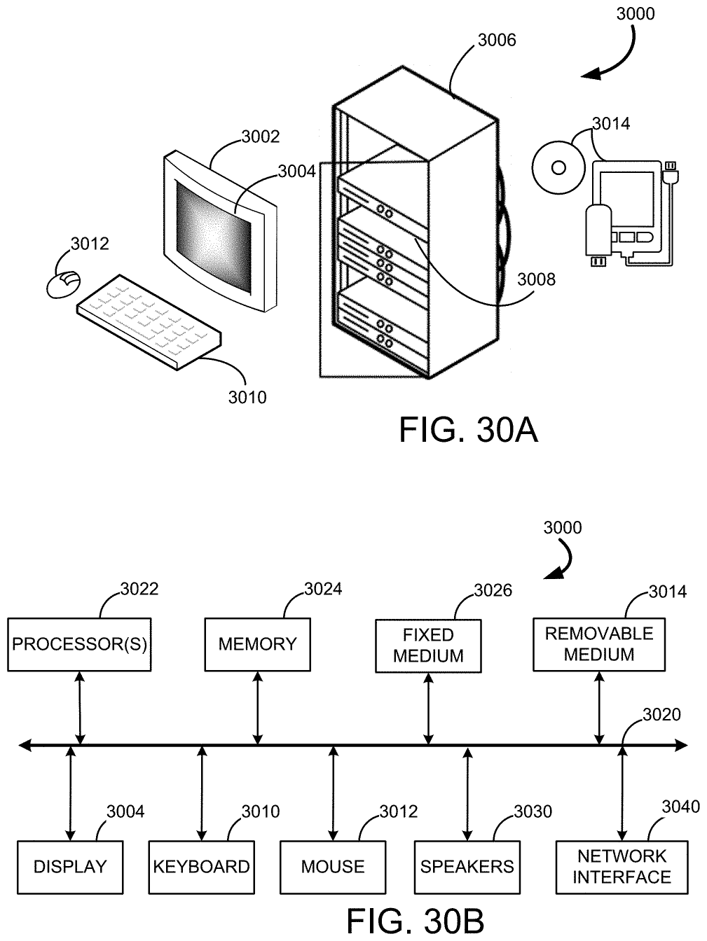

Now that the systems and methods for the mental exercises and improvements in mental acuity have been provided, attention shall now be focused upon apparatuses capable of executing the above functions in real-time. To facilitate this discussion, FIGS. 30A and 30B illustrate a Computer System 3000, which is suitable for implementing embodiments of the present invention. FIG. 30A shows one possible physical form of the Computer System 3000. Of course, the Computer System 3000 may have many physical forms ranging from a printed circuit board, an integrated circuit, and a small handheld device up to a huge super computer. Computer system 3000 may include a Monitor 3002, a Display 3004, a Housing 3006, server blades including one or more storage Drives 3008, a Keyboard 3010, and a Mouse 3012. Medium 3014 is a computer-readable medium used to transfer data to and from Computer System 3000.

FIG. 30B is an example of a block diagram for Computer System 3000. Attached to System Bus 3020 are a wide variety of subsystems. Processor(s) 3022 (also referred to as central processing units, or CPUs) are coupled to storage devices, including Memory 3024. Memory 3024 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable form of the computer-readable media described below. A Fixed Medium 3026 may also be coupled bi-directionally to the Processor 3022; it provides additional data storage capacity and may also include any of the computer-readable media described below. Fixed Medium 3026 may be used to store programs, data, and the like and is typically a secondary storage medium (such as a hard disk) that is slower than primary storage. It will be appreciated that the information retained within Fixed Medium 3026 may, in appropriate cases, be incorporated in standard fashion as virtual memory in Memory 3024. Removable Medium 3014 may take the form of any of the computer-readable media described below.

Processor 3022 is also coupled to a variety of input/output devices, such as Display 3004, Keyboard 3010, Mouse 3012 and Speakers 3030. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, motion sensors, brain wave readers, or other computers. Processor 3022 optionally may be coupled to another computer or telecommunications network using Network Interface 3040. With such a Network Interface 3040, it is contemplated that the Processor 3022 might receive information from the network, or might output information to the network in the course of performing the above-described promotion offer generation and redemption. Furthermore, method embodiments of the present invention may execute solely upon Processor 3022 or may execute over a network such as the Internet in conjunction with a remote CPU that shares a portion of the processing.

Software is typically stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory in this disclosure. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

In operation, the computer system 3000 can be controlled by operating system software that includes a file management system, such as a medium operating system. One example of operating system software with associated file management system software is the family of operating systems known as Windows® from Microsoft Corporation of Redmond, Wash., and their associated file management systems. Another example of operating system software with its associated file management system software is the Linux operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is, here and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may, thus, be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, Glasses with a processor, Headphones with a processor, Virtual Reality devices, a processor, distributed processors working together, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer (or distributed across computers), and when read and executed by one or more processing units or processors in a computer (or across computers), cause the computer(s) to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A training method for enhancing the mental capabilities of a user, the method comprising:
collecting contextual information regarding a user;
presenting the user with a cognitive task, wherein the cognitive task includes a stimuli and the task specifies a desired response to the stimuli;
collecting feedback from the user regarding the cognitive task;
assessing the performance of the user in relation to the cognitive task;
generating guidance for the user based upon the contextual information, the feedback, and the assessed performance.

2. The training method of claim 1, wherein the stimuli is presented at a plurality of random inter-stimulus intervals (ISI), and wherein the random inter-stimulus intervals are substantially between a reaction-reset interval and an attention-sustaining interval.

3. The training method of claim 1, wherein the guidance is generated by a rule-based engine.

4. The training method of claim 1, wherein the guidance is generated by an AI model.

5. The training method of claim 1, wherein the feedback includes EEG data.

6. The training method of claim 1, wherein the context includes biometric data collected from the user, environmental conditions, date, and time.

7. The training method of claim 1, wherein the guidance includes resource documents and progression to an advanced cognitive task.

8. The training method of claim 1, wherein a device is used to present the cognitive task to the user.

9. The training method of claim 8, wherein where the device is one of a smartphone, a tablet, a personal computer, a VR headset, and an augmented reality headset.

10. The training method of claim 8, wherein the device is integrated into physical exercise equipment.

11. The training method of claim 1, wherein the stimuli is a near-threshold stimuli.

12. A computer product embodied within non-transitory memory that, when executed on a computer system, performs the steps of:
collecting contextual information regarding a user;
presenting the user with a cognitive task, wherein the cognitive task includes a stimuli and the task specifies a desired response to the stimuli;
collecting feedback from the user regarding the cognitive task;
assessing the performance of the user in relation to the cognitive task;
generating guidance for the user based upon the contextual information, the feedback, and the assessed performance.

13. The non-transitory computer product of claim 12, wherein the stimuli is presented at a plurality of random inter-stimulus intervals (ISI), and wherein the random inter-stimulus intervals are substantially between a reaction-reset interval and an attention-sustaining interval.

14. The non-transitory computer product of claim 12, wherein the guidance is generated by a rule based engine.

15. The non-transitory computer product of claim 12, wherein the guidance is generated by an AI model.

16. The non-transitory computer product of claim 11, wherein the feedback includes EEG data.

17. The non-transitory computer product of claim 12, wherein the context includes biometric data collected from the user, environmental conditions, date, and time.

18. The non-transitory computer product of claim 12, wherein the guidance includes resource documents and progression to an advanced cognitive task.

19. The non-transitory computer product of claim 12, wherein a device is used to present the cognitive task to the user.

20. The non-transitory computer product of claim 19, wherein where the device is one of a smartphone, a tablet, a personal computer, a VR headset, and an augmented reality headset.

21. The non-transitory computer product of claim 19, wherein the device is integrated into physical exercise equipment.
